SEQUENCE LISTING (58 sequences)

SEQ ID NO: 1 - DNA, 52 bp, Homo sapiens
  misc_feature (27)..(27): n is c or t
gtgggttgacgttctcagacacaggtncccatcggccacatatttgaggtct

SEQ ID NO: 2 - DNA, 52 bp, Homo sapiens
  misc_feature (27)..(27): n is c or t
tgaactggaagactccgatgtgttttnctcagtccctcccactttgacactc

SEQ ID NO: 3 - DNA, 52 bp, Homo sapiens
  misc_feature (27)..(27): n is c or t
gggtcacagagctagcaaaaggggaancaggatttgaacctgagtctgtttg

SEQ ID NO: 4 - DNA, 52 bp, Homo sapiens
  misc_feature (27)..(27): n is a or g
tgaccacaaggcaacaagtttagggtntgaagctatcattttgaggaaggta

SEQ ID NO: 5 - DNA, 52 bp, Homo sapiens
  misc_feature (27)..(27): n is c or g
tgaggtcaggagttcgagaccaggctngccaacatggtgaaaccctatctct

SEQ ID NO: 6 - DNA, 52 bp, Homo sapiens
  misc_feature (27)..(27): n is c or t
cattttttcagtgagtgtaaaacatancccagaatttgaagatttactgtgg

SEQ ID NO: 7 - DNA, 52 bp, Homo sapiens
  misc_feature (27)..(27): n is a or g
ccactatcactacagacctcatggacnttaaaagaataataagggaatactg

SEQ ID NO: 8 - DNA, 52 bp, Homo sapiens
  misc_feature (27)..(27): n is a or t
gttcttttgtaccttgatcaaaattcntttgggtatacttatgtggttctgt

SEQ ID NO: 9 - DNA, 52 bp, Homo sapiens
  misc_feature (27)..(27): n is c or g
cagagagaaagggagctgaggggatgnagaggctgcccactgagggcagggg

SEQ ID NO: 10 - DNA, 52 bp, Homo sapiens
  misc_feature (27)..(27): n is c or t
caggaatatgaggctctgctcaagaantgaggtgtgacgaaggacttgaagg

SEQ ID NO: 11 - DNA, 52 bp, Homo sapiens
  misc_feature (27)..(27): n is c or t
gggaattttgtgtattttgttctctgntgggccctcagtgcacaggacagtg

SEQ ID NO: 12 - DNA, 52 bp, Homo sapiens
  misc_feature (27)..(27): n is a or c
atcagaggtcaagcacagagttttcanggttcacctatgttgtagcatacat

SEQ ID NO: 13 - DNA, 52 bp, Homo sapiens
  misc_feature (27)..(27): n is a or g
cctaaatatgatttcctaaatcatacngacatatttccttgggagctataca

SEQ ID NO: 14 - DNA, 52 bp, Homo sapiens
  misc_feature (27)..(27): n is c or g
aaaaatagaagaattatctgggcatgntggtgggtgcctgcagctccagctg

SEQ ID NO: 15 - DNA, 52 bp, Homo sapiens
  misc_feature (27)..(27): n is a or g
catggtctcagtctgtagcccaagctngagcatagtagtggcacaatcgcca

SEQ ID NO: 16 - DNA, 52 bp, Homo sapiens
  misc_feature (27)..(27): n is c or t
aaatcactggcaattgttggggggagnggctgttgttttgttctttttgggg

SEQ ID NO: 17 - DNA, 52 bp, Homo sapiens
  misc_feature (27)..(27): n is a or g
gaaaacaccagtagaagaaccagctgnccacctgagaatcttgagaaattac

SEQ ID NO: 18 - DNA, 52 bp, Homo sapiens
  misc_feature (27)..(27): n is g or t
cttttgttttcctttctgtgagcaatntcacccaaattggaaccatgctgta

SEQ ID NO: 19 - DNA, 52 bp, Homo sapiens
  misc_feature (27)..(27): n is c or t
cttcctgacatcactccaatgtcctgnttctgtggttacatcttccgctaat

SEQ ID NO: 20 - DNA, 52 bp, Homo sapiens
  misc_feature (27)..(27): n is a or c
tattcatttttccaacaagcatcctgncccaggtcgctctgtctgtctcaat

SEQ ID NO: 21 - DNA, 52 bp, Homo sapiens
  misc_feature (27)..(27): n is a or t
agtaagtcttgtatttcacctcctggnggtaaatattttttaacaatttgtc

SEQ ID NO: 22 - DNA, 52 bp, Homo sapiens
  misc_feature (27)..(27): n is c or t
ctgaacatacatcatatgaagaggcangcttatgatctgcacctgcgtctgg

SEQ ID NO: 23 - DNA, 52 bp, Homo sapiens
  misc_feature (27)..(27): n is c or g
tcgtaagcagcctgggagatgtgggcntaagctttggtgaggatgagagtct

SEQ ID NO: 24 - DNA, 52 bp, Homo sapiens
  misc_feature (27)..(27): n is a or t
ctcaattgaggaagaatagccttttcnacaaatggtgtttggaccaattgga

SEQ ID NO: 25 - DNA, 52 bp, Homo sapiens
  misc_feature (27)..(27): n is a or g
gcttcaaaaacatctgcccccaactcntttgtgctttcaccactgctaggaa

SEQ ID NO: 26 - DNA, 52 bp, Homo sapiens
  misc_feature (27)..(27): n is c or t
gctcctttgccgagtaacataagatangcacagggtccagggatgcggtgct

SEQ ID NO: 27 - DNA, 52 bp, Homo sapiens
  misc_feature (27)..(27): n is c or t
aactggatcttccccaggagtcatcantgtagcagtggggttgggttttaaa

SEQ ID NO: 28 - DNA, 52 bp, Homo sapiens
  misc_feature (27)..(27): n is a or g
agagggcacagccagtgtggtcaggtnggagcagagggaaggggtagcaggt

SEQ ID NO: 29 - DNA, 52 bp, Homo sapiens
  misc_feature (27)..(27): n is a or c
agaacaaatgctgtatgattccccctncatgaggtgctgagagaagtcaaat

SEQ ID NO: 30 - DNA, 52 bp, Homo sapiens
  misc_feature (27)..(27): n is a or g
gacaagaggaagtaggaagagaagaanaggatggagacaatgcttgacaatt

SEQ ID NO: 31 - DNA, 52 bp, Homo sapiens
  misc_feature (27)..(27): n is a or g
tgggcttgggaggaggccctgggactncagcacccgcacccacctgtagacg

SEQ ID NO: 32 - DNA, 52 bp, Homo sapiens
  misc_feature (27)..(27): n is a or g
ctgagagaagtcaaattcctagaaacngacgtgtctaaatatttgccggggt

SEQ ID NO: 33 - DNA, 52 bp, Homo sapiens
  misc_feature (27)..(27): n is c or t
tcatataacaatatgaaagccagaganagctcgtctgagacacagatgaaca

SEQ ID NO: 34 - DNA, 52 bp, Homo sapiens
  misc_feature (27)..(27): n is c or t
gcacgtttcatttgtttattgatttcngcctgattactccagaaggtaatta

SEQ ID NO: 35 - DNA, 20 bp, Artificial
  synthetic sequence
ggtggcctgagtttcagttc

SEQ ID NO: 36 - DNA, 18 bp, artificial
  synthetic sequence
cccggtcatgtctgtgtc

SEQ ID NO: 37 - DNA, 19 bp, artificial
  synthetic sequence
gtgggcagcctctgcattc

SEQ ID NO: 38 - DNA, 27 bp, artificial
  synthetic sequence
caaatacataaatagcgactgggtgac

SEQ ID NO: 39 - DNA, 19 bp, artificial
  synthetic sequence
cttccgccagtcatgcaac

SEQ ID NO: 40 - DNA, 20 bp, artificial
  synthetic sequence
agcaggcaccttgaaatgtc

SEQ ID NO: 41 - DNA, 20 bp, artificial
  synthetic sequence
ggtggcctgagtttcagttc

SEQ ID NO: 42 - DNA, 20 bp, artificial
  synthetic sequence
tgcccagaggccaatatttc

SEQ ID NO: 43 - DNA, 20 bp, artificial
  synthetic sequence
ccttcgtcacacctcaattc

SEQ ID NO: 44 - DNA, 20 bp, artificial
  synthetic sequence
ggaaggtatgttcccaagag

SEQ ID NO: 45 - DNA, 20 bp, artificial
  synthetic sequence
gagcaggtggaatcctcttg

SEQ ID NO: 46 - DNA, 18 bp, artificial
  synthetic sequence
cccggtcatgtctgtgtc

SEQ ID NO: 47 - DNA, 19 bp, artificial
  synthetic sequence
gtgggcagcctctgcattc

SEQ ID NO: 48 - DNA, 18 bp, artificial
  synthetic sequence
agcagaagcgactcttcc

SEQ ID NO: 49 - DNA, 18 bp, artificial
  synthetic sequence
ggctaacctgtgcctttg

SEQ ID NO: 50 - DNA, 19 bp, artificial
  synthetic sequence
ggagctgggagaggatatg

SEQ ID NO: 51 - DNA, 18 bp, artificial
  synthetic sequence
ctgacgctgaaggttctg

SEQ ID NO: 52 - DNA, 27 bp, artificial
  synthetic sequence
caaatacataaatagcgactgggtgac

SEQ ID NO: 53 - DNA, 19 bp, artificial
  synthetic sequence
cttccgccagtcatgcaac

SEQ ID NO: 54 - DNA, 20 bp, artificial
  synthetic sequence
tcaagtgatcctcccaactc

SEQ ID NO: 55 - DNA, 20 bp, artificial
  synthetic sequence
cctggatgtgattgctcaag

SEQ ID NO: 56 - DNA, 19 bp, artificial
  synthetic sequence
ggtggagaatgacactctg

SEQ ID NO: 57 - DNA, 18 bp, artificial
  synthetic sequence
tgagctgctggaacaaag

SEQ ID NO: 58 - DNA, 20 bp, artificial
  synthetic sequence
agcaggcaccttgaaatgtc

(19) United States
(12) Patent Application Publication
(10) Pub. No.: US 2011/0165124 A1
(43) Pub. Date: Jul. 7, 2011

(54) METHODS FOR DIAGNOSING OR PREDICTING HEPATITIS C OUTCOME IN HCV INFECTED PATIENTS

(75) Inventors: Pierre-Yves Bochud, Pully (CH); Andri Rauch, Bern (CH)

(73) Assignees: Centre Hospitalier Universitaire Vaudois, Lausanne (CH); University of Bern, Bern (CH)

(21) Appl. No.: 12/846,356

(22) Filed: Jul. 29, 2010

Related U.S. Application Data
(60) Provisional application No. 61/282,538, filed on Feb. 26, 2010.

(30) Foreign Application Priority Data
Jul. 31, 2009 (CH) CH 01201/09

(51) Int. Cl. A61K 38/21; C12Q 1/68; A61P 31/14; A61P 31/12

(52) U.S. Cl. 424/85.7; 435/6.11; 424/85.4

(57) ABSTRACT

The present invention relates to in vitro methods of determining a susceptibility to non-response to a hepatitis C treatment or a susceptibility to spontaneous hepatitis C clearance in a subject infected with hepatitis C.

### CROSS-REFERENCE TO RELATED APPLICATIONS

This application claims the benefit of U.S. provisional application Ser. No. 61/282,538, filed Feb. 26, 2010, and CH 01201/09, filed Jul. 31, 2009, the entire disclosures of which are expressly incorporated herein by reference.

### FIELD OF THE INVENTION

The present invention relates to in vitro methods of determining a susceptibility to non-response to a hepatitis C treatment or a susceptibility to spontaneous hepatitis C clearance in a subject infected with hepatitis C.

### BACKGROUND OF THE INVENTION

Hepatitis C virus (HCV) is a single stranded RNA virus that infects chronically more than 200 million persons, that is ˜3% of the world population [1-4]. Acute infection with the hepatitis C Virus (HCV) induces a wide range of innate and adaptive immune responses that achieve a permanent control of HCV in 20-50% of persons [5]. Failure to clear the virus leads to chronic hepatitis C. Chronic infection is associated with significant morbidity and mortality, resulting mainly from the progression towards cirrhosis and hepatocellular carcinoma [6]. The current standard therapy of pegylated interferon and ribavirin (PEG-IFN/RBV) results in sustained response rates in 30-80% of chronically infected individuals [7-9].

Increasing evidence suggests that host genetic factors influence both the natural course of chronic hepatitis C infection and response to therapy [10-13]. In two cohorts of pregnant women infected under similar conditions from immunoglobulin preparations contaminated with a single strain of HCV, half spontaneously cleared the infection and half progressed to chronic hepatitis C [14, 15]. Among chronically infected patients, response to treatment differs, even between cases with similar HCV-RNA levels and identical genotypes [6, 7, 9]. The response rates are strongly associated with ethnicity and gender [16]. Previous reports revealed the influence of genetic polymorphisms of human leukocyte antigens (HLA) [10, 13], killer immunoglobulin-like receptors (KIRs) [17], cytokines (WO 00/08215), chemokines and interleukins as well as interferon-stimulated genes [18-22] on HCV infection outcomes.

Previous studies have used a candidate gene approach based on a priori knowledge of the potential role of a gene in HCV infection. However, previous data do not allow accurate prediction of spontaneous clearance or response to treatment [13].

Despite the above-mentioned approach, there is still a profound need to develop an effective predictive method of determining a susceptibility to non-response to an anti hepatitis C therapy in a subject suffering from chronic hepatitis C, or a susceptibility to spontaneous or non spontaneous hepatitis C clearance in a subject acutely infected with hepatitis C virus.

To date, no efficient methods or strategies have been developed to overcome this problem.

### SUMMARY OF THE INVENTION

This object has been achieved by providing a method of determining a susceptibility to non-response to a hepatitis C treatment in a subject suffering from chronic hepatitis C, said method comprising determining the presence or absence of at least one polymorphic marker in the IL28B/A and/or IL-29 locus in a nucleic acid sample isolated from a biological sample obtained from said subject.

A further object of the present invention is to provide a method of determining a susceptibility to non-spontaneous hepatitis C clearance in a subject infected with hepatitis C, said method comprising determining the presence or absence of at least one polymorphic marker within the IL28B/A and/or IL-29 locus in a nucleic acid sample isolated from a biological sample obtained from said subject.

The invention also provides a method for determining whether a human subject having hepatitis C may be at risk for resistance to hepatitis C treatment, said method comprising, detecting the presence or absence of at least one polymorphic marker in the IL28B/A and/or the IL-29 locus in a nucleic acid sample of the subject, wherein the presence of the at least one polymorphic marker indicates an increased risk for resistance to hepatitis C treatment in the subject.

The invention further provides a method for determining whether a human subject having hepatitis C may be at risk for being unable to clear hepatitis C spontaneously, said method comprising, detecting the presence or absence of at least one polymorphic marker in the IL28B/A and/or the IL-29 locus in a nucleic acid sample of the subject, wherein the presence of the at least one polymorphic marker indicates an increased risk that the subject may be unable to clear hepatitis C spontaneously.

In some embodiments, the methods of the present invention further comprise determining the HCV viral genotype of the subject, wherein the presence of HCV viral genotype 1 or 4 is an indication that the subject has an increased susceptibility to non-response to hepatitis C treatment.

Another object of the invention is to provide a method of treating a patient for chronic hepatitis C, comprising i) determining whether at least one of the patient's polymorphic markers is in the IL28B/A and/or IL-29 locus in a nucleic acid sample isolated from a biological sample obtained from said subject wherein said at least one of the patient's polymorphic markers is selected from the group comprising rs11879005, rs12975799, rs11083519, rs955155, rs12972991, rs12980275, rs8105790, rs11881222, rs10853727, rs8109886, rs8113007, rs8099917, rs7248668, rs16973285, rs10853728, rs4803223, rs12980602, rs4803224, rs664893, rs576832, rs11671087, rs251910, rs7359953, rs7359950, rs2099331, rs11665818, rs570880, rs503355, rs30461, rs194014, rs251903, rs12979175, rs39587, rs30480, ii) and treating the patient based upon whether the at least one of the patient's polymorphic markers is associated with increased susceptibility to non-response to hepatitis C treatment.

Still a further object of the present invention is a method of treating a patient for chronic hepatitis C, comprising i) determining whether at least one of the patient's polymorphic markers is in the IL28B/A and/or IL-29 locus in a nucleic acid sample isolated from a biological sample obtained from the patient wherein said at least one of the patient's polymorphic markers is selected from the group comprising rs11879005, rs12975799, rs11083519, rs955155, rs12972991, rs12980275, rs8105790, rs11881222, rs10853727, rs8109886, rs8113007, rs8099917, rs7248668, rs16973285, rs10853728, rs4803223, rs12980602, rs4803224, rs664893, rs576832, rs11671087, rs251910, rs7359953, rs7359950, rs2099331, rs11665818, rs570880, rs503355, rs30461, rs194014, rs251903, rs12979175, rs39587, rs30480, ii) determining the HCV viral genotype in a nucleic acid sample isolated from a biological sample obtained from said patient, iii) and treating the patient based upon whether the at least one of the patient's polymorphic markers and HVC viral genotype are associated with increased susceptibility to non-response to hepatitis C treatment.

This invention also provides a method of assessing a susceptibility to non-response to a hepatitis C treatment in a subject suffering from chronic hepatitis C, said method comprising: i) distinguishing in said subjects those having a susceptibility to non-response to a hepatitis C treatment by determining the presence or absence of at least one polymorphic marker in the IL28B/A and/or IL-29 locus in a nucleic acid sample isolated from a biological sample of said subject, the presence of the at least one polymorphic marker being an indication that said subject has an increased susceptibility to non-response to a hepatitis C treatment, ii) establishing a hepatitis C treatment regimen.

This invention also deals with a method of assessing a susceptibility to non-response to a hepatitis C treatment in a subject suffering from chronic hepatitis C, said method comprising: i) distinguishing in said subjects those having a susceptibility to non-response to a hepatitis C treatment by determining
the presence or absence of at least one polymorphic marker in the IL28B/A and/or IL-29 locus in a nucleic acid sample isolated from a biological sample obtained from said subject, the presence of the at least one polymorphic marker being an indication that said subject has an increased susceptibility to non-response to a hepatitis C treatment, and
the HCV viral genotype, the presence of genotype 1 or 4 being an indication that said subject has an increased susceptibility to non-response to a hepatitis C treatment,
   ii) establishing a hepatitis C treatment regimen.

The invention further provides a method of treating a patient for chronic hepatitis C, comprising determining whether at least one of the patient's polymorphic markers is in the IL28B/A and/or IL-29 locus in a nucleic acid sample isolated from a biological sample obtained from said patient, determining the HCV viral genotype in a sample from said patient, and treating the patient based upon whether the at least one of the patient's polymorphic markers and HCV viral genotype are associated with increased susceptibility to non-response to hepatitis C treatment.

The invention further provides a method of assessing a susceptibility to non-response to a hepatitis C treatment in a subject suffering from chronic hepatitis C, said method comprising distinguishing in said subject or subjects those having a susceptibility to non-response to a hepatitis C treatment by determining the presence or absence of at least one polymorphic marker in the IL28B/A and/or IL-29 locus in a sample of said subject, the presence of the at least one polymorphic marker being an indication that said subject has an increased susceptibility to non-response to a hepatitis C treatment, and establishing a hepatitis C treatment regimen based on the risk for increased susceptibility.

Also included in the invention is a method of assessing a susceptibility to non-response to a hepatitis C treatment in a subject suffering from chronic hepatitis C, said method comprising distinguishing in said subjects those having a susceptibility to non-response to a hepatitis C treatment by determining the presence or absence of at least one polymorphic marker in the IL28B/A and/or IL-29 locus in a nucleic acid sample isolated from a biological sample obtained from said subject, the presence of the at least one polymorphic marker being an indication that said subject has an increased susceptibility to non-response to a hepatitis C treatment, and the HCV viral genotype, the presence of genotype 1 or 4 being an indication that said subject has an increased susceptibility to non-response to a hepatitis C treatment, and further establishing a hepatitis C treatment regimen.

In one embodiment, the at least one of the patient's polymorphic markers is selected from the group comprising rs11879005, rs12975799, rs11083519, rs955155, rs12972991, rs12980275, rs8105790, rs11881222, rs10853727, rs8109886, rs8113007, rs8099917, rs7248668, rs16973285, rs10853728, rs4803223, rs12980602, rs4803224, rs664893, rs576832, rs11671087, rs251910, rs7359953, rs7359950, rs2099331, rs11665818, rs570880, rs503355, rs30461, rs194014, rs251903, rs12979175, rs39587, rs30480.

In one embodiment, the hepatitis C treatment is an interferon based treatment. In another embodiment, the interferon based treatment is selected from the group comprising IFNα, IFNλ or any pegylated-interferon. In yet another embodiment, the interferon based treatment is combined with ribavirin or any antiprotease drugs or any other antiviral drugs or any combination thereof.

The present invention also relates to a kit for determining a susceptibility to non-response to a hepatitis C treatment in a subject suffering from chronic hepatitis C in accordance with the invention, said kit comprising i) reagents for selectively detecting the presence or absence of at least one polymorphic marker in the IL28B/A and/or IL-29 locus in a nucleic acid sample isolated from a biological sample obtained from the subject and ii) instructions for use.

Also provided in the present invention is a kit for determining a susceptibility to non-spontaneous hepatitis C clearance in a subject infected with hepatitis C in accordance with the invention, said kit comprising i) reagents for selectively detecting the presence or absence of at least one polymorphic marker within the IL28B/A and/or IL-29 locus in a nucleic acid sample isolated from a biological sample obtained from the subject and ii) instructions for use.

In one embodiment, the reagents further comprise another primer, a set of primers, or an array of primers, directed to detect the viral genotype.

In one embodiment, the methods and compositions of the invention relate to a subject having both hepatitis C and HIV.

### BRIEF DESCRIPTION OF THE FIGURES

FIG. 1 represents a Manhattan plot. The P-values for all 2.5M imputed Single Nucleotide Polymorphisms (SNPs) are indicated (on −log10 scale).

FIG. 2 represents the distribution of genotypes in an infected population. (A) Genotypes containing the G-allele were reduced in individuals with spontaneous HCV clearance compared to chronic infection. (B) In the Swiss Hepatitis C Cohort Study (SCCS), there was an increasing frequency of the G allele across the three following groups of patients: those with spontaneous viral clearance<those with clearance following treatment (i.e. responders to treatment)<those with non-response to treatment.

FIG. 3 is a graphic representation of the P values of the different SNPs showing a concordant association pattern for both spontaneous clearance and non-response to treatment in the IL28B haplotype block (A) Haplotype blocks. The strongest genetic association is located in a haplotype block that is closest to the IL28B gene. (B) Genetic Association of SNPs with HCV in the IL28B/A and IL-29 locus. The strong association of SNPs located near the IL28B and IL28A loci was present for both endpoints, spontaneous Hepatitis C clearance and non response to interferon-based therapy.

### DETAILED DESCRIPTION OF THE INVENTION

The present invention concerns a method of determining a susceptibility to non-response to a hepatitis C treatment in a subject suffering from chronic hepatitis C, said method comprising determining the presence or absence of at least one polymorphic marker in the IL28B/A and/or IL-29 locus in a nucleic acid sample isolated from a biological sample obtained from said subject.

The present invention also concerns a method of determining a susceptibility to non-spontaneous hepatitis C clearance in a subject infected with hepatitis C, said method comprising determining the presence or absence of at least one polymorphic marker within the IL28B/A and/or IL-29 locus in a nucleic acid sample isolated from a biological sample obtained from said subject.

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components.

As used in the specification and claims, the singular form “a”, “an” and “the” include plural references unless the context clearly dictates otherwise.

As used herein “Sustained viral response” was defined as an undetectable viremia more than 24 weeks after treatment was terminated.

As used herein, “at least one” means “one or more.”

As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of a hepatitis C treatment. However, in other embodiments, the subject can be a normal subject.

The terms “subject” or “patient” do not denote a particular age or sex. Thus, adult, infant and newborn subjects, whether male or female, are intended to be covered.

Alternatively, said subject or patient is co-infected with the human immunodeficiency virus (HIV), preferably HIV-1 or HIV-2.

As used herein the term “susceptibility” refers to the likelihood, for the subject, or a predisposition not to respond to hepatitis C treatment or to a predisposition, for the subject, to a non-spontaneous hepatitis C clearance.

An “allele”, as used herein, refers to one specific form of a genetic sequence or a single nucleotide position within a genetic sequence (such as a gene) within a cell, an individual or within a population, the specific form differing from other forms of the same gene in the sequence of at least one, and frequently more than one, variant sites within the sequence of the gene. The sequence may or may not be within a gene. The sequences at these variant sites that differ between different alleles are termed “variances”, “polymorphisms”, or “mutations”. At each autosomal specific chromosomal location or “locus”, an individual possesses two alleles, one inherited from one parent and one from the other parent, for example one from the mother and one from the father.

“Polymorphism,” as used herein, refers to the occurrence of two or more genetically determined alternative sequences or alleles in a population. A “polymorphic marker” or site is the locus at which divergence occurs. Preferred markers have at least two alleles, each occurring at a frequency of preferably greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, copy number variations (CNV) and insertion elements such as Alu. The first identified allelic form is arbitrarily designated as the reference form and other allelic forms are designated as alternative or variant alleles. The allelic form occurring most frequently in a selected population is sometimes referred to as the wildtype form. A diallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens. A particular kind of polymorphism, called a single nucleotide polymorphism, or SNP, is a small genetic change or variation that can occur within a person's DNA sequence. The genetic code is specified by the four nucleotide “letters” A (adenine), C (cytosine), T (thymine), and G (guanine). SNP variation occurs when a single nucleotide, such as an A, replaces one of the other three nucleotide letters—C, G, or T.

The term “hepatitis C virus” or “HCV” is used herein to define an RNA viral species of which pathogenic strains cause hepatitis C, also known as non-A, non-B hepatitis. Based on genetic differences between HCV isolates, the hepatitis C virus species is classified into six genotypes (1-6) with several subtypes within each genotype. Subtypes are further broken down into quasi species based on their genetic diversity. The preponderance and distribution of HCV genotypes varies globally. For example, in North America, genotype 1a predominates followed by 1b, 2a, 2b, and 3a. In Europe, genotype 1b is predominant followed by 2a, 2b, 2c, and 3a. Genotypes 4 and 5 are found almost exclusively in Africa. The viral genotype is clinically important in determining potential response to interferon-based therapy and the required duration of such therapy. Genotypes 1 and 4 are generally less responsive to interferon-based treatment than are the other genotypes (2, 3, 5 and 6). It is to be noted that genotypes 5 and 6 are rare in the population.

“Hepatitis C” is an infectious disease affecting the liver, caused by the hepatitis C virus (HCV). The infection is often asymptomatic, but once established, chronic hepatitis C infection can progress to scarring of the liver (fibrosis), and advanced scarring (cirrhosis) which is generally apparent after many years. In some cases, those with cirrhosis will go on to develop liver failure or other complications of cirrhosis, including liver cancer.

“Chronic hepatitis C” is defined as infection with the hepatitis C virus persisting for more than six months. Clinically, it is often asymptomatic (without symptoms) and it is mostly discovered accidentally. The natural course of chronic hepatitis C varies considerably from one person to another. Although almost all people infected with HCV have evidence of inflammation on liver biopsy, the rate of progression of liver scarring (fibrosis) shows significant variability among individuals. Accurate estimates of the risk over time are difficult to establish because of the limited time that tests for this virus have been available.

Soon after the hepatitis C virus (HCV) was identified, a number of cross-sectional studies in people with antibodies to the virus demonstrated that some appeared to show “spontaneous hepatitis C clearance”, while others maintained a state of viraemia. Since then, a number of investigators have endeavoured to characterize the pathogenesis of hepatitis C infection, including the rate, time course and predictors of spontaneous viral clearance. Estimates of clearance rates have ranged from 10 to 50%, and the duration of time to clearance has been found to be as long as 3 years in some cases. Authoritative clinical reviews have generally quoted clearance rates as low as 10-15%.

“Non-spontaneous hepatitis C clearance” refers herein to a situation where a subject would not present spontaneous clearance, such that the infection would evolve into chronic hepatitis C. Non-spontaneous hepatitis C clearance refers to failed spontaneous hepatitis C clearance. For the sake of clarity, it does not refer to a treatment-induced clearance.

“IL28B/A and/or IL-29 locus” generally refers, in humans, to a genomic DNA region located within a 80 kb region in the long arm of chromosome 19 encoding three cytokine genes, i.e. IL28B, IL28A and IL29 (which belong to the IFNλ family). These three genes have several exons, 5 for IL-28 (also referred to as IFNλ1) and 6 exons for IL-28A (IFNλ2) and IL-28B (IFNλ3). They encode 20 kDa secreted monomeric proteins. It has recently been reported that IL28B, IL28A and IL29 cytokines could be an interesting substitute to IFNα for the treatment of HCV-infected patients who are or become resistant to IFNα ([38]).

In the case of a method of determining a susceptibility to non-response to a hepatitis C treatment (or resistance to hepatitis C treatment) in a subject suffering from chronic hepatitis C, according to the present invention, the presence of the at least one polymorphic marker is an indication that said subject has an increased susceptibility to non-response to a hepatitis C treatment.

On the other hand, in the case of a method of determining a susceptibility to non-spontaneous hepatitis C clearance in a subject infected with hepatitis C, according to the present invention, then the presence of the at least one polymorphic marker is an indication that said subject has an increased susceptibility to non-spontaneous hepatitis C clearance.

A number of methods are available for analyzing the presence or absence of at least one single nucleotide polymorphism (SNP), which can be applied to the IL28B/A and/or IL-29 locus in a nucleic acid sample isolated from a biological sample obtained from said subject. Assays for detection of polymorphisms or mutations fall into several categories, including but not limited to direct sequencing assays, fragment polymorphism assays, hybridization assays, and computer based data analysis. Protocols and commercially available kits or services for performing multiple variations of these assays are available. In some embodiments, assays are performed in combination or in hybrid (e.g., different reagents or technologies from several assays are combined to yield one assay). The following assays are useful in the present invention, and are described in relationship to detection of the various SNP found in the IL28B/A and/or IL-29 locus.

In one aspect of the present invention, SNPs are detected using a direct sequencing technique. In these assays, DNA samples are first isolated from a subject using any suitable method. In some embodiments, the region of interest is cloned into a suitable vector and amplified by growth in a host cell (e.g., a bacterium). In other embodiments, DNA in the region of interest is amplified using the Polymerase Chain Reaction (PCR).

Following amplification, DNA in the region of interest (e.g., the region containing the SNP) is sequenced using any suitable method, including but not limited to manual sequencing using radioactive marker nucleotides, or automated sequencing. The results of the sequencing are displayed using any suitable method. The sequence is examined and the presence or absence of a given SNP is determined.

In one aspect of the present invention, SNPs are detected using a PCR-based assay. In some embodiments, the PCR assay comprises the use of oligonucleotide primers (“primers”) to amplify a fragment containing the repeat polymorphism of interest. Amplification of a target polynucleotide sequence may be carried out by any method known to the skilled artisan. See, for instance, [41] and [42]. Amplification methods include, but are not limited to, PCR including real time PCR (RT-PCR), strand displacement amplification [43]; [44], strand displacement amplification using Phi29 DNA polymerase (U.S. Pat. No. 5,001,050), transcription-based amplification [45], self-sustained sequence replication (“3SR”) [46]; [47], the Q.beta. replicase system ([48]; [49]), nucleic acid sequence-based amplification (“NASBA”) ([50]), the repair chain reaction (“RCR”) ([50], supra), and boomerang DNA amplification (or “BDA”) ([50]). PCR is the preferred method of amplifying the target polynucleotide sequence.

PCR may be carried out in accordance with techniques known by the skilled artisan. In general, PCR involves, first, treating a nucleic acid sample (e.g., in the presence of a heat stable DNA polymerase) with a pair of amplification primers. One primer of the pair hybridizes to one strand of a target polynucleotide sequence. The second primer of the pair hybridizes to the other, complementary strand of the target polynucleotide sequence. The primers are hybridized to their target polynucleotide sequence strands under conditions such that an extension product of each primer is synthesized which is complementary to each nucleic acid strand. The extension product synthesized from each primer, when it is separated from its complement, can serve as a template for synthesis of the extension product of the other primer. After primer extension, the sample is treated to denaturing conditions to separate the primer extension products from their templates. These steps are cyclically repeated until the desired degree of amplification is obtained.

The amplified target polynucleotide may be used in one of the detection assays described elsewhere herein to identify the GT-repeat polymorphism present in the amplified target polynucleotide sequence.

In one aspect of the present invention, SNPs are detected using a fragment length polymorphism assay. In a fragment length polymorphism assay, a unique DNA banding pattern based on cleaving the DNA at a series of positions is generated using an enzyme (e.g., a restriction endonuclease). DNA fragments from a sample containing a polymorphism will have a different banding pattern than wild type.

In one aspect of the present invention, fragment sizing analysis is carried out using the Beckman Coulter CEQ 8000 genetic analysis system, a method well-known in the art for microsatellite polymorphism determination.

In one aspect of the present invention, SNPs are detected using a restriction fragment length polymorphism assay (RPLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining and compared to controls (wild-type).

In one aspect, polymorphisms are detected using a CLEAVASE fragment length polymorphism assay (CFLP; Third Wave Technologies, Madison, Wis.: see e.g., U.S. Pat. No. 5,888,750). This assay is based on the observation that, when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions.

The region of interest is first isolated, for example, using PCR. Then, DNA strands are separated by heating. Next, the reactions are cooled to allow intrastrand secondary structure to form. The PCR products are then treated with the CLEAVASE I enzyme to generate a series of fragments that are unique to a given polymorphism. The CLEAVASE enzyme treated PCR products are separated, detected (e.g., by agarose gel electrophoresis), visualized (e.g., by ethidium bromide staining) and compared to controls (wild-type).

In other aspects of the present invention, SNPs are detected by hybridization assay. In a hybridization assay, the presence or absence of a given polymorphism or mutation is determined based on the ability of the DNA from the sample to hybridize to a complementary DNA molecule (e.g., an oligonucleotide probe). A variety of hybridization assays using a variety of technologies for hybridization and detection are available. A description of a selection of assays is provided below.

In a preferred aspect, the hybridized nucleic acids are detected by detecting one or more labels attached to the sample nucleic acids. The labels may be incorporated by any of a number of means well known to those of skill in the art. In one embodiment, the label is simultaneously incorporated during the amplification step in the preparation of the sample nucleic acids.

Thus, for example, polymerase chain reaction (PCR) with labeled primers or labeled nucleotides will provide a labeled amplification product. In another embodiment, transcription amplification using a labeled nucleotide (e.g. fluorescein-labeled UTP and/or CTP) incorporates a label into the transcribed nucleic acids.

Alternatively, a label may be added directly to the original nucleic acid sample (e.g., mRNA, polyA mRNA, cDNA, genomic DNA etc.) or to the amplification product after the amplification is completed. Means of attaching labels to nucleic acids are well known to those of skill in the art and include, for example, nick translation or end-labeling (e.g. with a labeled RNA) by kinasing the nucleic acid and subsequent attachment (ligation) of a nucleic acid linker joining the sample nucleic acid to a label (e.g., a fluorophore). In another embodiment label is added to the end of fragments using terminal deoxytransferase (TdT).

Detectable labels suitable for use in the present invention include any composition detectable by spectroscopic, photochemical, biochemical, immunochemical, electrical, optical or chemical means. Useful labels in the present invention include, but are not limited to: biotin for staining with labeled streptavidin conjugate; anti-biotin antibodies; magnetic beads (e.g., Dynabeads™); fluorescent, dyes (e.g., fluorescein, Texas Red, rhodamine, green fluorescent protein, and the like); radiolabels (e.g., ³H, ¹²⁵I, ³⁵S, ¹⁴C, or ³²P); phosphorescent labels; enzymes (e.g., horse radish peroxidase, alkaline phosphatase and others commonly used in an ELISA); and calorimetric labels such as colloidal gold or colored glass or plastic (e.g., polystyrene, polypropylene, latex, etc.) beads.

Means of detecting such labels are well known to those of skill in the art. Thus, for example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

The label may be added to the target nucleic acid(s) prior to, or after the hybridization. So-called “direct labels” are detectable labels that are directly attached to or incorporated into the target nucleic acid prior to hybridization. In contrast, so-called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting, labeled hybridized nucleic acids. See Tijssen, 1993, Laboratory Techniques in Biochemistry and Molecular Biology, Vol. 24; Hybridization with Nucleic Acid Probes, which is hereby incorporated by reference in its entirety for all purposes.

In one aspect, hybridization of a probe to the sequence of interest (e.g., polymorphism like SNP) is detected directly by visualizing a bound probe (e.g., a Northern or Southern assay; See e.g., Ausabel et al. (Eds.), 1991, Current Protocols in Molecular Biology, John Wiley & Sons, NY). In these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., agarose gel electrophoresis) and transferred to a membrane. A labeled (e.g., by incorporating a radionucleotide) probe or probes specific for the mutation being detected is allowed to contact the membrane under a condition of low, medium, or high stringency conditions. Unbound probe is removed and the presence of binding is detected by visualizing the labeled probe.

In one aspect of the present invention, SNPs are detected using a DNA chip hybridization assay. In this assay, a series of oligonucleotide probes are affixed to a solid support. The oligonucleotide probes are designed to be unique to a given single nucleotide polymorphism. The DNA sample of interest is contacted with the DNA “chip” and hybridization is detected.

In some embodiments, the DNA chip assay is a GeneChip (Affymetrix, Santa Clara, Calif.; see e.g., U.S. Pat. No. 6,045,996) assay. The GeneChip technology uses miniaturized, high-density arrays of oligonucleotide probes affixed to a “chip”. Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated from a biological sample obtained from the subject, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In another aspect, a DNA microchip containing electronically captured probes (Nanogen, San Diego, Calif.) is utilized (see e.g., U.S. Pat. No. 6,068,818). Through the use of microelectronics, Nanogen's technology enables the active movement and concentration charged molecules to and from designated test sites on its semiconductor microchip. DNA capture probes unique to a given polymorphism or mutation are electronically placed at, or “addressed” to, specific sites on the microchip. Since DNA has a strong negative charge, it can be electronically moved to an area positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound nonspecifically bound DNA from each site, the polarity or charge of the site is reversed to negative, thereby forcing any unbound or nonspecifically bound DNA back into solution away from the capture probes. A laser-based fluorescence scanner is used to detect binding.

In still another aspect, an array technology based upon the segregation of fluids on a flat surface (chip) by differences in surface tension (ProtoGene, Palo Alto, Calif.) is utilized (see e.g., U.S. Pat. No. 6,001,311). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on an X/Y translation stage under a set of four piezoelectric nozzles, one for each of the four standard DNA bases. The translation stage moves along each of the rows of the array, and the appropriate reagent is delivered to each of the reaction site. For example, the A amidite is delivered only to the sites where amidite A is to be coupled during that synthesis step and so on. Common reagents and washes are delivered by flooding the entire surface followed by removal by spinning.

DNA probes unique for the polymorphism of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other aspects, a “bead array” is used for the detection of SNP's (Illumina, San Diego, Calif.; see e.g., PCT Publications WO99/67641 and WO00/39587, each of which is herein incorporated by reference). Illumina uses a bead array technology that combines fiber optic bundles and beads that self-assemble into an array. Each fiber optic bundle contains thousands to millions of individual fibers depending on the diameter of the bundle. The beads are coated with an oligonucleotide specific for the detection of a given polymorphism or mutation. Batches of beads are combined to form a pool specific to the array. To perform an assay, the bead array is contacted with a prepared subject sample (e.g., DNA). Hybridization is detected using any suitable method like Enzymatic Detection of Hybridization

In some aspects of the present invention, genomic profiles are generated using an assay that detects hybridization by enzymatic cleavage of specific structures (INVADER assay, Third Wave Technologies; see e.g., U.S. Pat. No. 6,001,567). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the dequenched fluorescein labeled product may he detected using a standard fluorescence plate reader.

The INVADER assay detects specific mutations and polymorphisms in unamplified genomic DNA. The isolated DNA sample is contacted with the first probe specific either for a polymorphism/mutation or wild type sequence and allowed to hybridize. Then a secondary probe, specific to the first probe, and containing the fluorescein label, is hybridized and the enzyme is added. Binding is detected using a fluorescent plate reader and comparing the signal of the test sample to known positive and negative controls.

In some aspects, hybridization of a bound probe is detected using a TaqMan assay (PE Biosystems, Foster City, Calif.; see e.g., U.S. Pat. No. 5,962,233). The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can he monitored with a fluorimeter.

In some aspects, a MassARRAY system (Sequenom, San Diego, Calif.) is used to detect polymorphisms (see e.g., U.S. Pat. No. 6,043,031). DNA is isolated from blood samples using standard procedures. Next, specific DNA regions containing the polymorphism of interest are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization-Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than 0.0001 second, enabling samples to be analyzed in a total of 3-5 second including repetitive data collection. The SpectroTYPER software then calculates, records, compares and reports, the genotypes at the rate of three seconds per sample.

Usually, the “nucleic acid sample” of the invention is isolated from a biological sample obtained from the subject, such as whole blood, serum, semen, saliva, tears, urine, fecal material, sweat, buccal smears, skin, and biopsies of muscle, liver, brain tissue, nerve tissue and hair. The nucleic acid sample may be a portion of a gene, a regulatory sequence, genomic DNA, cDNA, and RNA including mRNA, mi RNA and rRNA).

Genomic DNA samples are usually amplified before being brought into contact with a probe. Genomic DNA can be obtained from any biological sample. Amplification of genomic DNA containing a SNP generates a single species of nucleic acid if the individual from whom the sample was obtained is homozygous at the polymorphic site, or two species of nucleic acid if the individual is heterozygous.

RNA samples also are often subject to amplification. In this case, amplification is typically preceded by reverse transcription. Amplification of all expressed mRNA can be performed as described in, for example, in [39] and [40] which are hereby incorporated by reference in their entirety. Amplification of an RNA sample from a diploid sample can generate two species of target molecules if the individual providing the sample is heterozygous at a polymorphic site occurring within the expressed RNA, or possibly more if the species of the RNA is subjected to alternative splicing. Amplification generally can be performed using the polymerase chain reaction (PCR) methods known in the art. Nucleic acids in a target sample can be labeled in the course of amplification by inclusion of one or more labeled nucleotides in the amplification mixture. Labels also can be attached to amplification products after amplification (e.g., by end-labeling). The amplification product can be RNA or DNA, depending on the enzyme and substrates used in the amplification reaction.

The genotype of an individual polymorphism comprises the sum of at least two alleles and may be homozygous (i.e. comprising identical alleles) or heterozygous (i.e. comprising different alleles).

In some aspect of the invention, the isolated nucleic acid sample of the present invention can be produced or synthesized using conventional nucleic acid synthesis or by recombinant nucleic acid methods known in the art (2001, Molecular Cloning: A Laboratory Manual, Cold Spring Harbor Laboratory Press, New York) and Ausubel et al. (2001, Current Protocols in Molecular Biology, Green & Wiley, New York).

Surprisingly the Inventors of the present invention have shown that the presence of the at least one polymorphic marker in the IL28B/A and/or IL-29 locus in a nucleic acid sample isolated from a biological sample obtained from a subject suffering from chronic hepatitis C is an indication that said subject has an increased susceptibility to non-response to a hepatitis C treatment.

Surprisingly also the inventors of the present invention have shown that the presence of the least one polymorphic marker in the IL28B/A and/or IL-29 locus in a nucleic acid sample isolated from a biological sample obtained from a subject infected with hepatitis C is an indication that said subject has an increased susceptibility to non-spontaneous hepatitis C clearance.

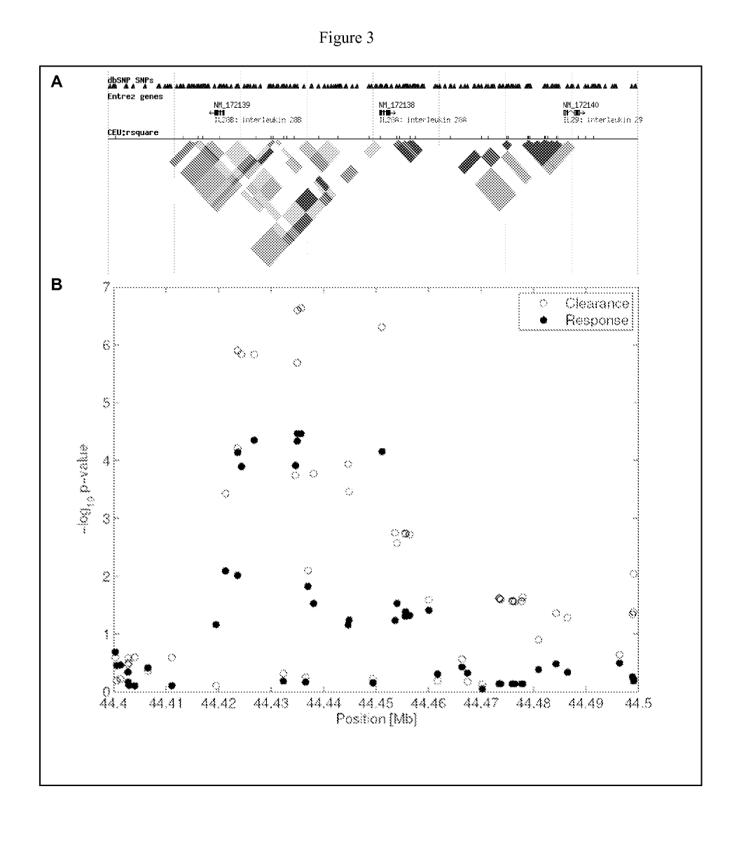

Preferably the at least one SNP of the invention is located on human chromosome 19 within a region comprising about 80 kb. Haplotype blocks mapping showed (FIG. 3) a strong genetic association between the SNPs and i) on one hand a susceptibility to non-response to a hepatitis C treatment in a subject suffering from chronic hepatitis C, ii) and on the other hand an increased susceptibility to non-spontaneous hepatitis C clearance.

More preferably, the at least one SNP of the invention is located in a nucleic acid segment essentially consisting in the DNA regions flanking the SNP selected from the group comprising SEQ ID No 1, SEQ ID No 2, SEQ ID No 3, SEQ ID No 4, SEQ ID No 5, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18, SEQ ID No 19, SEQ ID No 20, SEQ ID No 21, SEQ ID No 22, SEQ ID No 23, SEQ ID No 24, SEQ ID No 25, SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, (as listed in Table 1).

The present invention also contemplates determining the presence or absence of at least one, i.e. one or more as defined supra, i.e. a combination of, single nucleotide polymorphism (SNP) in the IL28B/A and/or IL-29 locus in a nucleic acid sample isolated from a biological sample obtained from said subject.

Preferably, the polymorphic marker is a polymorphic site associated with at least one SNP selected from the group comprising rs11879005, rs12975799, rs11083519, rs955155, rs12972991, rs12980275, rs8105790, rs11881222, rs10853727, rs8109886, rs8113007, rs8099917, rs7248668, rs16973285, rs10853728, rs4803223, rs12980602, rs4803224, rs664893, rs576832, rs11671087, rs251910, rs7359953, rs7359950, rs2099331, rs11665818, rs570880, rs503355, rs30461, rs194014, rs251903, rs12979175, rs39587, rs30480. Most preferably, the SNP is selected from the group comprising—G/T for rs8099917, G/G for rs8099917, C/G for rs576832, C/C for rs576832, G/A for rs12980275 or G/G for rs12980275.

Preferably also, the at least one polymorphic marker is a polymorphic site being in complete or strong linkage disequilibrium with at least one SNP selected from the group comprising rs11879005, rs12975799, rs11083519, rs955155, rs12972991, rs12980275, rs8105790, rs11881222, rs10853727, rs8109886, rs8113007, rs8099917, rs7248668, rs16973285, rs10853728, rs4803223, rs12980602, rs4803224, rs664893, rs576832, rs11671087, rs251910, rs7359953, rs7359950, rs2099331, rs11665818, rs570880, rs503355, rs30461, rs194014, rs251903, rs12979175, rs39587, rs30480.

For example, when said rs8099917 allele G is present on one chromosome (or one of the two allelic positions) it confers the heterozygous genotype G/T. In contrast, when present on the two chromosomes (or allelic position) it confers the homozygous genotype G/G.

In one aspect, the nucleic acid sample useful for the determination of the viral genotype and the nucleic acid sample useful for the determination of the polymorphism, as described herein, are isolated from the same biological sample obtained from the subject. The biological sample is then prepared on on hand for the isolation of the nucleic acid sample useful for determining the presence or absence of the at least one polymorphic marker of the invention and on the other hand for determining the HCV viral genotype.

In another aspect, the nucleic acid sample useful for the determination of the viral genotype and the nucleic acid sample useful for the determination of the polymorphism, as described herein, are isolated from two different biological samples obtained from the subject. In this case, the first biological sample is then prepared for the isolation of the nucleic acid sample useful for determining the presence or absence of the at least one polymorphic marker of the invention whereas the second biological sample is prepared for the isolation of the nucleic acid sample useful for determining the HCV viral genotype.

These two biological samples can be of same nature (e.g. whole blood in the two cases) or different (e.g. whole blood and liver biopsy).

The HCV nucleic acid, usually RNA, to be analyzed is generally isolated, reverse transcribed into cDNA and amplified, for example, by PCR as described in WO 96/14839 and WO 97/01603. Any other techniques known in the art can be applied.

“Linkage disequilibrium” (LD) describes a situation in which some combinations of alleles or genetic markers occur more or less frequently in a population than would be expected from a random formation of haplotypes from alleles based on their frequencies. When a particular allele at one locus is found together on the same chromosome with a specific allele at a second locus-more often than expected if the loci were segregating independently in a population-the loci are in disequilibrium. This concept of LD is formalized by one of the earliest measures of disequilibrium to be proposed (symbolized by D). D, in common with most other measures of LD, quantifies disequilibrium as the difference between the observed frequency of a two-locus haplotype and the frequency it would be expected to show if the alleles are segregating at random. Adopting the standard notation for two adjacent loci-A and B, with two alleles (A, a and B, b) at each locus-the observed frequency of the haplotype that consists of alleles A and B is represented by PAB. Assuming the independent assortment of alleles at the two loci, the expected halotype frequency is calculated as the product of the allele frequency of each of the two alleles, or PA×PB, where PA is the frequency of allele A at the first locus and PB is the frequency of allele B at the second locus. So, one of the simplest measures of disequilibrium is D=PAB−PA×PB. LD is created when a new mutation occurs on a chromosome that carries a particular allele at a nearby locus, and is gradually eroded by recombination. Recurrent mutations can also lessen the association between alleles at adjacent loci. The importance of recombination in shaping patterns of LD is acknowledged by the moniker of “linkage”. The extent of LD in populations is expected to decrease with both time (t) and recombinational distance (r, or the recombination fraction) between markers. Theoretically, LD decays with time and distance according to the following formula, where D0 is the extent of disequilibrium at some starting point and Dt, is the extent of disequilibrium t generation later: Dt=(1−r)tD0

A wide variety of statistics have been proposed to measure the amount of LD, and these have different strengths, depending on the context. Although the measure D has the intuitive concepts of LD, its numerical value is of little use for measuring the strength of and comparing levels of LD. This is due to the dependence of D on allele frequencies. The two most common measures are the absolute value of D′ and r2.

The absolute value of D′ is determined by dividing D by its maximum possible value, given the allele frequencies at the two loci. The case of D′=1 is known as complete LD. Values of D′<1 indicate that the complete ancestral LD has been disrupted. The magnitude of values of D′<1 has no clear interpretation. Estimates of D′ are strongly inflated in small samples. Therefore, statistically significant values of D′ that are near one provide a useful indication of minimal historical recombination, but intermediate values should not be used for comparisons of the strength of LD between studies, or to measure the extent of LD.

The measure r2 is in some ways complementary to D′. r2 is equal to D2 divided by the product of the allele frequencies at the two loci. Hill and Roberson deduced that E [r2]=1/1+4Nc where c is the recombination rate in morgans between the two markers and N is the effective population size. This equation illustrates two important properties of LD. First, expected levels of LD are a function of recombination. The more recombination between two sites, the more they are shuffled with respect to one another, decreasing LD. Second, LD is a function of N, emphasizing that LD is a property of populations.

In the present application, strong linkage disequilibrium presents a correlation termed r2 of at least 0.6 and/or a D′ of 0.5 with said SNPs in the HapMap European dataset and/or in the population experimentally analyzed by the Inventors.

In accordance with the present invention, if said rs8099917 allele G or rs576832 C is present (in one or two exemplars), this is an indication that a subject suffering from chronic hepatitis C has an increased susceptibility to non-response to a hepatitis C treatment. Presence of two exemplars of rs8099917 allele G or rs576832 C, instead of one allele, compared to no G or C allele (respectively), further increases the risk of non-response to a hepatitis C treatment.

Similarly, if said rs8099917 allele G or rs576832 C is present (in one or two exemplars), this is an indication that a subject infected with hepatitis C has an increased susceptibility to non-spontaneous hepatitis C clearance (or to evolve to chronic hepatitis C). Presence of two exemplars of rs8099917 allele G or rs576832 C, instead of one allele, compared to no G or C allele (respectively), further increases the risk of non-spontaneous clearance.

On another hand, patients who carried the risk allele G of rs12980275 were more likely to be non-responders than the other patients (OR=1.99, 95% CI 1.57-2.54, P=1.74E−8). This association was still significant after adjustment for relevant covariates (age, sex, HCV genotype, fibrosis severity status and HCV viral load, adjusted P=4.61E−09). Similarly, patients who carried the A/G and G/G genotypes were more likely to be non responders than A/A carriers (OR=2.65 [95% CI: 2.18-3.18], P=2.67E−7, adjusted P=9.90E−8 for A/G; OR=3.68, [95% CI: 2.77-4.88], P=4.05E−6, adjusted P=1.13E−5 for G/G, using A/A as a reference).

It has also been found that the association between the presence of SNPs and the susceptibility to non response to hepatitis C treatment or to non spontaneous clearance was observed both in HCV mono-infected and in HCV/HIV co-infected individuals

TABLE 1

| SNPs | SEQ ID N0 | Flanking DNA region |
|---|---|---|
| rs30461 | SEQ ID N^{o} 1 | GTGGGTTGACGTTCTCAGACACAGGT[C/T]CCCATCGGCCACATATTTGAGGTCT |
| rs30480 | SEQ ID N^{o} 2 | TGAACTGGAAGACTCCGATGTGTTTT[C/T]CTCAGTCCCTCCCACTTTGACACTC |
| rs39587 | SEQ ID N^{o} 3 | GGGTCACAGAGCTAGCAAAAGGGGAA[C/T]CAGGATTTGAACCTGAGTCTGTTTG |
| rs194014 | SEQ ID N^{o} 4 | TGACCACAAGGCAACAAGTTTAGGGT[A/G]TGAAGCTATCATTTTGAGGAAGGTA |
| rs251903 | SEQ ID N^{o} 5 | TGAGGTCAGGAGTTCGAGACCAGGCT[C/G]GCCAACATGGTGAAACCCTATCTCT |
| rs251910 | SEQ ID N^{o} 6 | CATTTTTTCAGTGAGTGTAAAACATA[C/T]CCCAGAATTTGAAGATTTACTGTGG |
| rs503355 | SEQ ID N^{o} 7 | CCACTATCACTACAGACCTCATGGAC[A/G]TTAAAAGAATAATAAGGGAATACTG |
| rs570880 | SEQ ID N^{o} 8 | GTTCTTTTGTACCTTGATCAAAATTC[A/T]TTTGGGTATACTTATGTGGTTCTGT |
| rs576832 | SEQ ID N^{o} 9 | CAGAGAGAAAGGGAGCTGAGGGGATG[C/G]AGAGGCTGCCCACTGAGGGCAGGGG |
| rs664893 | SEQ ID N^{o} 10 | CAGGAATATGAGGCTCTGCTCAAGAA[C/T]TGAGGTGTGACGAAGGACTTGAAGG |
| rs955155 | SEQ ID N^{o} 11 | GGGAATTTTGTGTATTTTGTTCTCTG[C/T]TGGGCCCTCAGTGCACAGGACAGTG |
| rs2099331 | SEQ ID N^{o} 12 | ATCAGAGGTCAAGCACAGAGTTTTCA[A/C]GGTTCACCTATGTTGTAGCATACAT |
| rs4803223 | SEQ ID N^{o} 13 | CCTAAATATGATTTCCTAAATCATAC[A/G]GACATATTTCCTTGGGAGCTATACA |
| rs4803224 | SEQ ID N^{o} 14 | AAAAATAGAAGAATTATCTGGGCATG[C/G]TGGTGGGTGCCTGCAGCTCCAGCTG |
| rs7248668 | SEQ ID N^{o} 15 | CATGGTCTCAGTCTGTAGCCCAAGCT[A/G]GAGCATAGTAGTGGCACAATCGCCA |
| rs7359950 | SEQ ID N^{o} 16 | AAATCACTGGCAATTGTTGGGGGGAG[C/T]GGCTGTTGTTTTGTTCTTTTTGGGG |
| rs7359953 | SEQ ID N^{o} 17 | GAAAACACCAGTAGAAGAACCAGCTG[A/G]CCACCTGAGAATCTTGAGAAATTAC |
| rs8099917 | SEQ ID N^{o} 18 | CTTTTGTTTTCCTTTCTGTGAGCAAT[G/T]TCACCCAAATTGGAACCATGCTGTA |
| rs8105790 | SEQ ID N^{o} 19 | CTTCCTGACATCACTCCAATGTCCTG[C/T]TTCTGTGGTTACATCTTCCGCTAAT |
| rs8109886 | SEQ ID N^{o} 20 | TATTCATTTTTCCAACAAGCATCCTG[A/C]CCCAGGTCGCTCTGTCTGTCTCAAT |
| rs8113007 | SEQ ID N^{o} 21 | AGTAAGTCTTGTATTTCACCTCCTGG[A/T]GGTAAATATTTTTTAACAATTTGTC |
| rs10853727 | SEQ ID N^{o} 22 | CTGAACATACATCATATGAAGAGGCA[C/T]GCTTATGATCTGCACCTGCGTCTGG |
| rs10853728 | SEQ ID N^{o} 23 | TCGTAAGCAGCCTGGGAGATGTGGGC[C/G]TAAGCTTTGGTGAGGATGAGAGTCT |
| rs11083519 | SEQ ID N^{o} 24 | CTCAATTGAGGAAGAATAGCCTTTTC[A/T]ACAAATGGTGTTTGGACCAATTGGA |
| rs11665818 | SEQ ID N^{o} 25 | GCTTCAAAAACATCTGCCCCCAACTC[A/G]TTTGTGCTTTCACCACTGCTAGGAA |
| rs11671087 | SEQ ID N^{o} 26 | GCTCCTTTGCCGAGTAACATAAGATA[C/T]GCACAGGGTCCAGGGATGCGGTGCT |
| rs11879005 | SEQ ID N^{o} 27 | AACTGGATCTTCCCCAGGAGTCATCA[C/T]TGTAGCAGTGGGGTTGGGTTTTAAA |
| rs11881222 | SEQ ID N^{o} 28 | AGAGGGCACAGCCAGTGTGGTCAGGT[A/G]GGAGCAGAGGGAAGGGGTAGCAGGT |
| rs12972991 | SEQ ID N^{o} 29 | AGAACAAATGCTGTATGATTCCCCCT[A/C]CATGAGGTGCTGAGAGAAGTCAAAT |
| rs12975799 | SEQ ID N^{o} 30 | GACAAGAGGAAGTAGGAAGAGAAGAA[A/G]AGGATGGAGACAATGCTTGACAATT |
| rs12979175 | SEQ ID N^{o} 31 | TGGGCTTGGGAGGAGGCCCTGGGACT[A/G]CAGCACCCGCACCCACCTGTAGACG |
| rs12980275 | SEQ ID N^{o} 32 | CTGAGAGAAGTCAAATTCCTAGAAAC[A/G]GACGTGTCTAAATATTTGCCGGGGT |
| rs12980602 | SEQ ID N^{o} 33 | TCATATAACAATATGAAAGCCAGAGA[C/T]AGCTCGTCTGAGACACAGATGAACA |
| rs16973285 | SEQ ID N^{o} 34 | GCACGTTTCATTTGTTTATTGATTTC[C/T]GCCTGATTACTCCAGAAGGTAATTA |

It is to be noted that the SNPs noted in Table 1 between brackets are in no particular order relating to wildtype vs risk (minor) alleles, and are thus not indicative or limitative in this regard.

Generally, the hepatitis C treatment is an interferon based treatment. More preferably, the interferon based treatment is selected from the group comprising IFNα, IFNλ or any pegylated-interferon. Usually, said interferon based treatment is combined with ribavirin. Alternative combinations may include antiprotease drugs or other antiviral drugs.

Further encompassed in the present invention is a method of assessing a susceptibility to non-response to a hepatitis C treatment in a subject suffering from chronic hepatitis C, said method comprising: i) distinguishing in said subjects those having a susceptibility to non-response to a hepatitis C treatment by determining the presence or absence of at least one polymorphic marker in the IL28B/A and/or IL-29 locus in a nucleic acid sample isolated from a biological sample obtained from said subject, the presence of the at least one polymorphic marker being an indication that said subject has an increased susceptibility to non-response to a hepatitis C treatment, ii) establishing a hepatitis C treatment regimen.

The determination of the polymorphism in a subject suffering from chronic hepatitis C will enable the physician to establish the best hepatitis C treatment regimen for said subject (nature, dose and duration of hepatitis C treatment and/or other antiviral drugs). For example, if the above method reveals that at least one SNP is present in the IL28B/A and/or IL-29 locus in a nucleic acid sample obtained from said subject, indicating that said subject has an increased susceptibility to non-response to a hepatitis C treatment then this subject can be considered as good candidate for newer treatment strategies (such as therapy with higher doses of currently available drugs, longer treatment duration with currently available drugs and/or newer drugs).

Additionally, the Inventors have shown that a subject infected with HCV genotype 1 or 4 that carry at least one SNP in the IL28B/A and/or IL-29 locus of the invention, particularly in homozygosis, will have a very low probability of treatment induced clearance (i.e. “response to treatment” or “treatment success”), as shown in both Tables 4 and 5. Table 4 gives more specific data regarding the distribution of each genotype (TT, GT and GG) in the infected population while Table 5 considers the presence or absence of a risk allele. It is simply to be noted that Tables 4 and 5 are based on slightly different numbers of patients in the various groups. For example, Table 5 shows that among patients infected with genotypes 1 or 4, treatment failure occurred in 72% of risk-allele carriers infected compared to only 38% of non-carriers (OR=6.54 [95% CI: 4.65-9.201, P=3.70E−8). From another point of view, it is to be noted that treatment failure occurred in only 16% of patients with both low risk parameters (i. e. those infected with viral genotype 2 or 3 and where rs8099917 G allele is absent from the IL28 B locus), compared to 72% among those with both high risk parameters (i.e. those infected with viral genotype 1 or 4 and where rs8099917 G allele is present in the IL28 B locus) (OR 18.89 [95% CI: 12.87-27.71], P=1.84E−14).

These results also show an association between genetic variations in the IL28B/A locus and response to therapy (e.g. interferon based treatment) among subjects infected with HCV genotypes 2 or 3 (OR=3.32 [95% CI: 2.21-4.99], P=3.27E−3). The strength of this association is nevertheless lower than in the case of subjects infected with HCV genotypes 1 or 4. These showings demonstrate that the knowledge of both the viral genotype and the host polymorphisms are important to predict response to treatment.

Therefore, another aspect of the present invention comprises the combined determinations of the viral genotype, and of the determination of the polymorphism as described herein, in a subject suffering from chronic hepatitis C, so as to more finely assess the susceptibility to non-response to a hepatitis C treatment or susceptibility to non-spontaneous clearance of HCV infected subjects.

These combined determinations can occur concomitantly or not. If not concomitant, the viral genotype can be assessed first, and then, after a determined time, the determination of the polymorphism as described herein occurs. It is also envisioned that the determination of the polymorphism as described herein occurs first, and then, after a determined time, the viral genotype is assessed.

Usually, the determined time, which is the time or duration lapsed between the determination of the viral genotype and the determination of the polymorphism (and vice versa) can be comprised between a few seconds and several years.

Also encompassed in the present invention is a kit for determining a susceptibility to non-response to a hepatitis C treatment in a subject suffering from chronic hepatitis C in accordance with the present invention, said kit comprising i) reagents for selectively detecting the presence or absence of at least one single nucleotide polymorphism (SNP) in the IL28B/A and/or IL-29 locus in a nucleic acid sample isolated from a biological sample obtained from the subject and ii) instructions for use.

Further encompassed in the present invention is a kit for determining a susceptibility to non-spontaneous hepatitis C clearance in a subject infected with hepatitis C in accordance with the present invention, said kit comprising i) reagents for selectively detecting the presence or absence of at least one single nucleotide polymorphism (SNP) in the IL28B/A and/or IL-29 locus in a nucleic acid sample isolated from a biological sample obtained from the subject and ii) instructions for use.

Alternatively, the reagents used in the kits comprise an isolated nucleic acid, preferably a primer, a set of primers, or an array of primers, as described elsewhere herein. The primers may be fixed to a solid substrate. The kits may further comprise a control target nucleic acid and primers. One skilled in the art will, without undue experiments, be able to select the primers in accordance with the usual requirements. The isolated nucleic acids of the kit may also comprise a molecular label or tag.

Usually, the primer, set of primers, or array of primers, are directed to detect the presence or absence of at least one single nucleotide polymorphism (SNP) in the IL28B/A and/or IL-29 locus.

The presence or absence of at least one single nucleotide polymorphism (SNP) in the IL28B/A locus may for example, but not exclusively, be determined using a set of PCR primers or sequencing primers selected from those disclosed in Table 6 (SEQ Ids No 35 to 58).

In addition to the primers, set of primers, or array of primers, directed to detect the presence or absence of at least one single nucleotide polymorphism (SNP) in the IL28B/A and/or IL-29 locus from a nucleic acid sample isolated from a biological sample obtained from a subject, the reagents of the kit may comprise, for example, an other primer, set of primers, or array of primers, directed to separately detect the viral genotype isolated from a biological sample obtained from a subject. These set of primers, or array of primers used are generally known in the art or may be readily generated knowing the usual requirements.

In additional embodiments, the kits of the present invention comprise various reagents, such as buffers, necessary to practice the methods of the invention, as known in the art.

These reagents or buffers may for example be useful to extract and/or purify the nucleic from the biological sample obtained from the subject.

The kit may also comprise all the necessary material such as microcentrifuge tubes necessary to practice the methods of the invention.

The invention further contemplates a method of treating a patient for chronic hepatitis C, comprising i) determining whether at least one of the patient's polymorphic markers is in the IL28B/A and/or IL-29 locus in a nucleic acid sample isolated from a biological sample obtained from said patient selected from the group comprising rs11879005, rs12975799, rs11083519, rs955155, rs12972991, rs12980275, rs8105790, rs11881222, rs10853727, rs8109886, rs8113007, rs8099917, rs7248668, rs16973285, rs10853728, rs4803223, rs12980602, rs4803224, rs664893, rs576832, rs11671087, rs251910, rs7359953, rs7359950, rs2099331, rs11665818, rs570880, rs503355, rs30461, rs194014, rs251903, rs12979175, rs39587, rs30480, ii) and treating the patient based upon whether the at least one of the patient's polymorphic markers is associated with increased susceptibility to non-response to hepatitis C treatment

Generally, the hepatitis C treatment is an interferon based treatment. More preferably, the interferon based treatment is selected from the group comprising IFNα, IFNλ or any pegylated-interferon. Usually, said interferon based treatment is combined with ribavirin. Alternative combinations may include antiprotease drugs or other antiviral drugs.

The invention also considers a method of determining a susceptibility to non-response or resistance to a Cytomegalovirus (CMV), Herpes simplex virus 1 or 2 (HSV-1 or HSV-2), hepatitis B virus (HBV) or Influenza viruses treatment, or spontaneous clearance in a subject infected with one or more of this or these viruses, said method comprising determining the presence or absence of at least one single nucleotide polymorphism (SNP) in the IL28B/A and/or IL-29 locus in nucleic acid sample isolated from a biological sample obtained from said subject.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this specification, each of which is incorporated herein by reference in its entirety.

The foregoing description will be more fully understood with reference to the following Examples. Such Examples, are, however, exemplary of methods of practising the present invention and are not intended to limit the scope of the invention.

### EXAMPLES

### Example 1

Patients & Methods

Patients were included within the framework of the Swiss Hepatitis C Cohort Study (SCCS) and the Swiss HIV Cohort Study (SHCS), two multicenter studies carried out at 8 major Swiss hospitals and their local affiliated centres [23]. Written informed consent including genetic testing was mandatory for inclusion, and the study was approved by all local ethical committees. Due to the different genetic predictors of Hepatitis C outcomes in racially diverse populations [10, 13, 24, 25], the inventors restricted the analysis to Caucasians. Of note only the genotypes 1, 2, 3 and 4 were represented in this study.

Spontaneous Hepatitis C Clearance

Chronic HCV infection was defined as HCV-seropositivity (using ELISA and confirmed by Immunoblot) and detectable HCV RNA by quantitative assays. Spontaneous Hepatitis C clearance was defined as HCV-seropositivity and undetectable HCV RNA before starting anti-HCV therapy. To avoid the fluctuations of HCV RNA levels during the first year of infection (reviewed in [26]), the inventors determined HCV RNA levels at least 1 year after the first documented positive HCV-serology.

Genotyping

Genotyping was done using the Illumina genomic platform at the National Centre of Competence in Research “Frontiers in Genetics” in Geneva, Switzerland, by using the Illumina Human1M-Duo BeadChip, HumanHap550 or Human600W-Quad. Genotype calling was performed using the default settings of the Beadstudio software. Calls with genotyping score below 0.2 were excluded from further analysis. SNPs with call rate below 90% and individuals with call rate below 95% were filtered out Imputation was carried out using MACH based on measured SNPs with >90% call rate, minor allele frequency (MAF)>1%, Hardy-Weinberg p-value>10⁻⁷. In the resulting imputed data, SNPs with low imputation accuracy (r2−hat<0.3) were ignored. Population stratification and relatedness was assessed using the ancestry principal components as described in [28]. One of each genetically related/identical individual pair (relatedness>0.25) was excluded from further analysis. The gender of each genotyped individual was assessed for concordance with clinical data. Association analysis was performed using logistic regression with exact maximum-likelihood estimation. Covariates such first two ancestry principal component values and Hepatitis B Virus infection status (defined as presence or absence of HBs-Ag) were included in the model. Bonferroni correction was used to control the family-wise error rate. One million independent tests were assumed [Han et al 2009 Plos Genetics] yielding the commonly used 5*10⁻⁸ threshold.

Influence of HIV-Coinfection

To take into account the potential influence of HIV-coinfection on spontaneous HCV clearance, HCV-monoinfected and co-infected individuals were first analysed separately and subsequently, the inventors genomewide meta-analyzed the two cohorts. Association signals obtained from each cohort were meta-analysed using inverse-variance weighting. Association analysis was performed using a logistic regression model with exact maximum-likelihood estimation. Covariates influencing the outcome in the univariate analysis (P<0.1), along with the first two ancestry principal components were included in the model. The inventors used a mild p-value cut-off as an inclusion criterion for covariates in order not to disregard potentially important factors. To account for the fact that different genotyping platforms were used, the inventors excluded any SNP whose allele frequency (among patients with chronic infection) was significantly different (Chi-square test, P<10−4) between any two platforms. Genomic control was applied to the genome-wide p-values yielding lambda=1.04 (for the mono-infected cohort), 1.02 (for the co-infected cohort).

These values suggested very mild inflation and confirmed that possible population stratification was sufficiently corrected by including the first two ancestry principal components in the models. Bonferroni correction was used to adjust for multiple testing; we used 5*10−8 as significance threshold.

### Example 2

Results

The study included 1142 HCV infected patients (726 mono-infected and 416 HIV co-infected), among whom 245 had spontaneous viral clearance and 897 had chronic infection (Table 3). Among chronically infected patients, 404 were assessable for response to pegylated-interferon alpha ribavirin combination therapy. As expected, the factors associated with spontaneous clearance included lower age (P<0.001), female sex (P<0.001) and active hepatitis B (P<0.001). The factors associated with response to treatment included lower age (P=0.05), female sex (0.03), viral genotype 2 or 3 (P<0.001), lower viral load (P<0.001) and limited fibrosis (P=0.02).

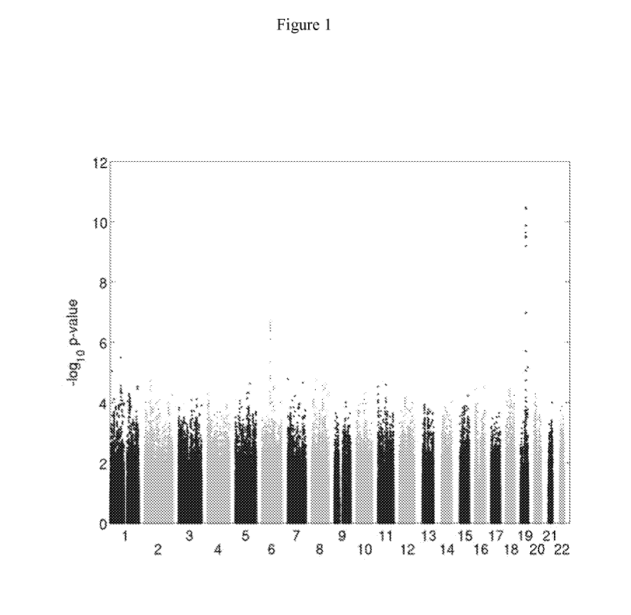

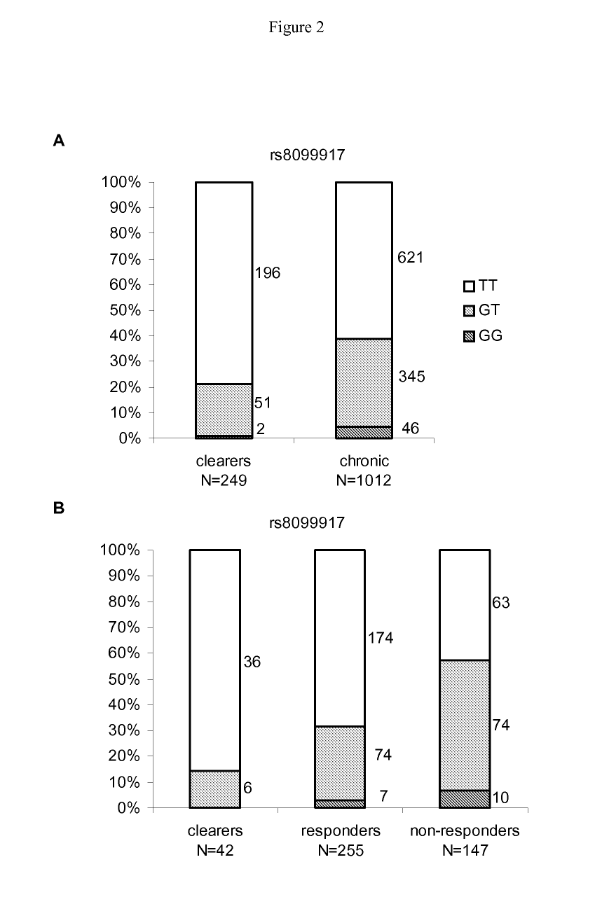

SNP rs8099917 is clearly associated with spontaneous hepatitis C clearance (FIG. 1). The frequencies of genotypes T/T (ancestral allele), G/T (heterozygous) and G/G (homozygous) were 0.79, 0.20 and 0.01 among patients with spontaneous clearance, versus 0.57, 0.38 and 0.05 among those with chronic infection, respectively (OR=0.38, 95% confidence interval [CI] 0.27-0.53, P=2.86E−08, under the additive mode, Table 4, FIG. 2A). The association of rs8099917 with spontaneous clearance was still present in a multivariate analyses accounting for age, sex, active hepatitis B and HCV risk groups (OR=0.38, 95% CI 0.28-0.53, P=6.81E−09, under the additive mode, Table 4).

The G allele of rs8099917 was also associated with non-response to pegylated interferon alpha ribavirin combination therapy (FIG. 2B). The frequencies of genotypes T/T, G/T and G/G were 0.68, 0.29 and 0.03 among patients with sustained viral response, versus 0.43, 0.50 and 0.07 among non-responders, respectively (OR=0.36, 95% CI 0.23-0.53, P=8.96E−07, under the dominant mode, OR=0.38, 95% CI 0.27-0.53, P=2.86E−06, under the additive mode, Table 4, FIG. 2B). The association of rs8099917 with non response to treatment was still present in a multivariate model accounting for age, sex, HCV RNA, HCV genotype, fibrosis stage and diabetes (OR=0.29, 95% CI 0.16-0.53, P=6.96E−05, under the additive mode). Overall, there was a progressive increase in the minor allele frequency of rs8099917 among patients with spontaneous clearance (0.07), sustained viral response (0.17) and non-response to treatment (0.32).

The rs8099917 SNP was located within a ˜80 kB region in the long arm of human chromosome 19 (in case the subject is a human) encoding three cytokine genes, i.e. IL28B, IL28A and IL29 (FIG. 3A). Haplotype blocks mapping showed that rs8099917 is part of a haplotypes block encompassing the whole IL-28B gene. A graphic representation of the P values of the different SNPs showed a concordant association pattern for both spontaneous clearance and response to treatment in the IL28B haplotype block (FIG. 3B).

The association was observed in HCV mono-infected (OR=2.49, 95% CI=1.64-3.79, P=1.96*10−5) as well as in HCV/HIV co-infected individuals (OR=2.16, 95% CI=1.47-3.18, P=8.24*10−5). In the case of spontaneous clearance, Table 4 shows a distribution of the various genotypes among the groups of mono-infected and co-infected patients and the associated frequency of clearance.

### Example 3

Pathogen Genetic Risk Determinant.

The inventors further assessed the joint contribution of host and pathogen genetic risk determinants. Patients were stratified in four groups, according to the viral genotypes (viral genotypes 2 or 3, versus viral genotypes 1 or 4) and host polymorphisms (host rs8099917 G risk allele carriers, versus non-carriers, Table 5). Treatment failure occurred in only 16% of patients with both low risk parameters, compared to 72% among those with both high risk parameters (OR=18.89 [95% CI: 12.87-27.71], P=1.84E−14, Table 5). Among patients infected with genotypes 1 or 4, treatment failure occurred in 72% of risk-allele carriers infected compared to only 38% of non-carriers (OR=6.54 [95% CI: 4.65-9.20], P=3.70E−8). Among patients infected with genotypes 2 or 3, treatment failure occurred in 25% of risk-allele carriers compared to 16% of non-carriers (OR=3.32 [95% CI: 2.21-4.99], P=3.27E−3). Again, Table 4 shows a distribution of the various genotypes (TT, TG, GG) among the groups of patients infected with viral genotypes 1&4 or 2&3, respectively, and the corresponding frequencies of response or non response to treatment.

Note for Table 2

It is to be noted that the wildtype/risk alleles of the SNPs given in Table 2 may be read on the same or the opposite strand compared to the corresponding sequence mentioned in Table 1.

Notes for Table 3

¹ SCCS stands for the Swiss Hepatitis C Cohort Study and SHCS for the Swiss HIV Cohort Study; 38 HIV-infected SCCS patients were analyzed together with SHCS patients.

² HBs antigen was missing in 352 mono-infected and 56 co-infected patients

³ HCV RNA at set point (for clearance endpoint) and before treatment (for treatment endpoint)

⁴ Heavy drinker was defined as use of more than 40 g alcohol per day for more than 5 years.

⁵ Biopsy data before treatment was missing in 128 patients. Severe fibrosis and inflammation were defined by a METAVIR score >2.

Notes for Table 4

¹ most likely model

² adjusted for age, sex, HBs AG and risk type

³ adjusted for age, sex, HCV RNA, HCV genotype, fibrosis stage and diabetes

⁴ adjusted for age, sex, HCV RNA, fibrosis stage and diabetes

Notes for Table 5

¹ adjusted for fibrosis stage, sex, age, baseline HCV viral load and the first two ancestry principal components.

² when analyzing genotypes 1 or 4 patients, treatment failure occurred in 72% of risk-allele carriers compared to only 38% of non-carriers (OR=6.54 [95% CI: 4.65-9.201, P=3.70E−8).

TABLE 2

| | | Allele | | |
|---|---|---|---|---|
| | | predisposing to | | |
| | | non-response | | |
| | | to treatment | | |
| | Wildtype | or non- | | |
| | allele | spontaneous | P-value for | P-value for |
| | (Major | clearance | Spontaneous | Response to |
| Rs Number | allele) | (minor allele) | clearance | treatment |
| rs11879005 | C | T | 4.52E−03 | 6.79E−01 |
| rs12975799 | G | A | 1.10E−02 | 7.89E−01 |
| rs11083519 | A | T | 1.19E−02 | 7.82E−01 |
| rs955155 | G | A | 9.54E−05 | 8.05E−03 |
| rs12972991 | A | C | 4.62E−07 | 9.68E−03 |
| rs12980275 | A | G | 8.97E−10 | 7.26E−05 |
| rs8105790 | T | C | 7.84E−09 | 1.26E−04 |
| rs11881222 | A | G | 1.15E−09 | 4.43E−05 |
| rs10853727 | T | C | 1.83E−01 | 6.50E−01 |
| rs8109886 | C | A | 6.84E−05 | 1.22E−04 |
| rs8113007 | A | T | 1.89E−09 | 4.58E−05 |
| rs8099917 | T | G | 3.48E−09 | 3.40E−05 |
| rs7248668 | G | A | 3.39E−09 | 3.41E−05 |
| rs16973285 | C | T | 3.29E−01 | 6.81E−01 |
| rs10853728 | G | C | 1.10E−03 | 1.51E−02 |
| rs4803223 | A | G | 4.68E−05 | 2.97E−02 |
| rs12980602 | T | C | 1.06E−04 | 7.00E−02 |
| rs4803224 | G | C | 4.39E−04 | 5.75E−02 |
| rs664893 | A | G | 3.00E−01 | 7.03E−01 |
| rs576832 | G | C | 1.19E−09 | 6.98E−05 |
| rs11671087 | T | C | 1.91E−02 | 5.82E−02 |
| rs251910 | C | T | 1.23E−02 | 2.96E−02 |
| rs7359953 | G | A | 1.78E−02 | 4.91E−02 |
| rs7359950 | C | T | 1.68E−02 | 4.14E−02 |
| rs2099331 | T | G | 1.79E−02 | 4.78E−02 |
| rs11665818 | G | A | 8.94E−02 | 3.89E−02 |
| rs570880 | A | T | 2.40E−01 | 3.72E−01 |
| rs503355 | C | T | 3.31E−01 | 8.97E−01 |
| rs30461 | A | G | 2.50E−01 | 4.12E−01 |
| rs194014 | G | A | 8.20E−02 | 3.31E−01 |
| rs251903 | G | C | 6.89E−02 | 4.59E−01 |
| rs12979175 | G | A | 1.78E−01 | 3.20E−01 |
| rs39587 | A | G | 5.26E−02 | 5.52E−01 |
| rs30480 | G | A | 3.77E−02 | 5.90E−01 |

TABLE 3
Patient's Demographic

| | HCV Mono-infected Patients (SCCS) | | | | | Co-infected Patients (SHCS)¹ | | | | | Response to Treatment (SCCS) | | | | |
|---|---|---|---|---|---|---|---|---|---|---|---|---|---|---|---|
| Characteristics | Spontaneous | | Chronic | | | Spontaneous | | Chronic | | | Sustained | | Non- | | |
| (N, proportion) | Clearance | | Infection | | P | Clearance | | Infection | | P | Responders | | responders | | P |
| N | 42 | | 684 | | | 207 | | 231 | | | 257 | | 147 | | |
| Age (median, IQR) | 37 | (9) | 45 | (15) | <0.001 | 39 | (8) | 39 | (10) | 0.3 | 44 | (16) | 47 | (13) | 0.05 |
| Male sex | 19 | (0.45) | 419 | (0.61) | 0.04 | 108 | (0.52) | 157 | (0.68) | <0.001 | 150 | (0.58) | 102 | (0.62) | 0.03 |
| White Ethnicity | 42 | (1.0) | 684 | (1.0) | | 207 | (1.0) | 231 | (1.0) | | 257 | (1.0) | 147 | (1.0) | |
| Risk group | | | | | | | | | | | | | | | |
| Drug use | 23 | (0.52) | 291 | (0.43) | Ref. | 160 | (0.77) | 176 | (0.76) | Ref. | 104 | (0.40) | 58 | (0.39) | Ref. |
| Transfusion | 4 | (0.09) | 136 | (0.19) | 0.09 | 2 | (0.01) | 4 | (0.02) | 0.5 | 46 | (0.18) | 28 | (0.19) | 0.8 |
| Invasive procedure | 9 | (0.21) | 170 | (0.24) | 0.4 | | | | | | 58 | (0.23) | 31 | (0.21) | 0.9 |
| Other/unknown | 7 | (0.17) | 87 | (0.13) | 0.9 | 45 | (0.22) | 51 | (0.22) | 0.9 | 49 | (0.19) | 30 | (0.20) | 0.7 |
| HBs antigen positive² | 2 | (0.07) | 6 | (0.02) | 0.06 | 21 | (0.11) | 8 | (0.04) | 0.01 | 5 | (0.03) | 1 | (0.01) | 0.4 |
| HIV positive | 0 | (0) | 0 | (0) | | 207 | (1.0) | 231 | (1.0) | | 8 | (0.04) | 0 | (0) | |
| HCV genotypes | | | | | | | | | | | | | | | |
| 1 | | | 334 | (0.49) | | | | 95 | (0.41) | | 81 | (0.32) | 91 | (0.62) | Ref. |
| 2 | | | 72 | (0.11) | | | | 7 | (0.03) | | 48 | (0.19) | 6 | (0.04) | <0.001 |
| 3 | | | 180 | (0.26) | | | | 69 | (0.30) | | 106 | (0.41) | 21 | (0.14) | <0.001 |
| 4 | | | 62 | (0.09) | | | | 24 | (0.10) | | 14 | (0.05) | 16 | (0.11) | 1.0 |
| Other/unknown | | | 36 | (0.05) | | | | 36 | (0.16) | | 8 | (0.03) | 13 | (0.09) | 0.4 |
| Log HCV RNA | | | 5.8 | (1.0) | | | | 6.1 | (1.2) | | 5.8 | (1.3) | 6.0 | (0.9) | <0.001 |
| (median, IQR)³ | | | | | | | | | | | | | | | |
| Heavy drinker⁴ | | | | | | | | | | | 83 | (0.33) | 57 | (0.40) | 0.2 |
| Liver biopsy⁵ | | | | | | | | | | | | | | | |
| Severe fibrosis | | | | | | | | | | | 50 | (0.29) | 45 | (0.43) | 0.02 |
| Severe inflammation | | | | | | | | | | | 35 | (0.20) | 20 | (0.20) | 0.9 |
| Steatosis | | | | | | | | | | | 120 | (0.69) | 73 | (0.75) | 0.7 |

TABLE 4
Association of Polymorphisms in the IL28A/B locus with Viral Clearance

| Group/ | rs8099917 | Clearance | | No Clearance | | Inheri- | Univariate | | Multivariate | |
|---|---|---|---|---|---|---|---|---|---|---|
| Endpoint | genotypes | N | Freq | N | Freq | tance | OR (95% CI) | P | OR (95% CI) | P |
| Mono-infected | TT | 36 | 0.86 | 380 | 0.56 | Rec. | 0.21 (0.09-0.50) | 4.84E−04 | 0.20 (0.08-0.49) | 4.45E−04 |
| Spontaneous | GT | 6 | 0.14 | 268 | 0.39 | Dom. | 0.21 (0.09-0.50) | 4.84E−04 | 0.20 (0.08-0.49) | 4.45E−04 |
| | GG | 0 | 0.00 | 34 | 0.05 | Add. | 0.24 (0.10-0.57) | 1.29E−03¹ | 0.21 (0.09-0.51) | 5.01E−04¹ |
| Co-infected | TT | 158 | 0.78 | 131 | 0.62 | Rec. | 0.20 (0.04-0.93) | 0.04 | 0.20 (0.04-0.95) | 0.04 |
| Spontaneous | GT | 43 | 0.21 | 72 | 0.34 | Dom. | 0.46 (0.30-0.70) | 3.43E−04 | 0.45 (0.29-0.70) | 4.52E−04 |
| | GG | 2 | 0.01 | 10 | 0.05 | Add. | 0.50 (0.32-0.77) | 1.88E−03¹ | 0.47 (0.31-0.70) | 2.13E−04¹ |
| All | TT | 194 | 0.79 | 511 | 0.57 | Rec. | 0.16 (0.04-0.66) | 0.01 | 0.16 (0.04-0.68) | 0.01² |
| Spontaneous | GT | 49 | 0.20 | 340 | 0.38 | Dom. | 0.35 (0.25-0.49) | 8.60E−10 | 0.36 (0.25-0.51) | 1.06E−08² |
| | GG | 2 | 0.01 | 44 | 0.05 | Add. | 0.38 (0.27-0.53) | 2.86E−08¹ | 0.38 (0.28-0.53) | 6.81E−09^{1,2} |

| | | Response to | | No Response to | | | | | | |
|---|---|---|---|---|---|---|---|---|---|---|
| Group/ | rs8099917 | Treatment | | Treatment | | Inheri- | Univariate | | Multivariate | |
| Endpoint | genotypes | N | Freq | N | Freq | tance | OR (95% CI) | P | OR (95% CI) | P |
| All | TT | 174 | 0.68 | 63 | 0.43 | Rec. | 0.39 (0.14-1.04) | 0.06 | 0.67 (0.15-2.95) | 0.6³ |
| Response to | GT | 74 | 0.29 | 74 | 0.50 | Dom. | 0.35 (0.23-0.53) | 8.96E−07¹ | 0.20 (0.10-0.40) | 7.52E−06^{1,3} |
| Treatment | GG | 7 | 0.03 | 10 | 0.07 | Add. | 0.36 (0.23-0.56) | 4.10E−06 | 0.29 (0.16-0.53) | 6.96E−05³ |
| SCCS Gen. 1 & 4 | TT | 67 | 0.72 | 42 | 0.39 | Rec. | 0.12 (0.01-0.95) | 0.04 | 0.76 (0.07-8.40) | 0.8⁴ |
| Response to | GT | 25 | 0.27 | 56 | 0.52 | Dom. | 0.25 (0.14-0.46) | 5.46E−06¹ | 0.29 (0.12-0.71) | 6.80E−03^{1,4} |
| Treatment | GG | 1 | 0.01 | 9 | 0.08 | Add. | 0.28 (0.15-0.51) | 4.17E−05 | 0.35 (0.15-0.81) | 1.36E−02⁴ |
| SCCS Gen. 2 & 3 | TT | 101 | 0.66 | 15 | 0.56 | Rec. | 0.66 (0.29-1.50) | 0.3 | | |
| Response to | GT | 47 | 0.31 | 12 | 0.44 | Dom. | 0.66 (0.29-1.50) | 0.3 | | |
| Treatment | GG | 6 | 0.04 | | | Add. | 0.58 (0.25-1.34) | 0.2 | | |

TABLE 5
Joint Analysis of Viral and Host Genetic Determinants of Treatment Response.

| | IL28B | Treatment | | Treatment | | Percent- | | |
|---|---|---|---|---|---|---|---|---|
| HCV | rs8099917 | Failure | | Success | | age of | | |
| Genotype | G allele | N | Freq. | N | Freq. | failure | OR (95% CI)¹ | P value |
| 2/3 | Absent | 29 | 0.13 | 154 | 0.40 | 16% | | Ref. |
| 2/3 | Present | 26 | 0.12 | 79 | 0.21 | 25% | 3.32 (2.21-4.99) | 3.27E−03 |
| 1/4 | Absent | 66 | 0.30 | 109 | 0.29 | 38%² | 3.23 (2.25-4.64) | 1.17E−03 |
| 1/4 | Present | 101 | 0.46 | 39 | 0.10 | 72%² | 18.89 (12.87-27.71) | 1.84E−14 |

TABLE 6
PCR and sequencing primers (IL 28B locus).

| Region amplified | PCR-Primers | Size | T° | SEQ ID |
|---|---|---|---|---|
| Promoter IL28B | F: 5′-GGTGGCCTGAGTTTCAGTTC-3′; | 1500 bp | 62° C. | No 35 |
| | R: 5′-CCCGGTCATGTCTGTGTC-3′ | | | No 36 |
| Exons and introns IL28B | F: 5′-GTGGGCAGCCTCTGCATTC-3′; | 1476 bp | 62° C. | No 37 |
| | R: 5′-CAAATACATAAATAGCGACTGGGTGAC-3′ | | | No 38 |
| 3′UTR IL28B | F: 5′-CTTCCGCCAGTCATGCAAC-3′; | 1450 bp | 65° C. | No 39 |
| | R: 5′-AGCAGGCACCTTGAAATGTC-3′ | | | No 40 |
| Region sequences | Sequencing-Primers | Size | T° | SEQ ID |
| Promoter part 1 | F: 5′-GGTGGCCTGAGTTTCAGTTC-3′; | 512 bp | 50° C. | No 41 |
| | R: 5′-TGCCCAGAGGCCAATATTTC-3′ | | | No 42 |
| Promoter part 2 | F: 5′-CCTTCGTCACACCTCAATTC-3′; | 581 bp | 50° C. | No 43 |
| | R: 5′-GGAAGGTATGTTCCCAAGAG-3′ | | | No 44 |
| Promoter part 3 | F: 5′-GAGCAGGTGGAATCCTCTTG-3′; | 529 bp | 50° C. | No 45 |
| | R: 5′-CCCGGTCATGTCTGTGTC-3′ | | | No 46 |
| Exons-Introns part 1 | F: 5′-GTGGGCAGCCTCTGCATTC-3′; | 540 bp | 50° C. | No 47 |
| | R: 5′-AGCAGAAGCGACTCTTCC-3′ | | | No 48 |
| Exons-Introns part 2 | F: 5′-GGCTAACCTGTGCCTTTG-3′; | 505 bp | 50° C. | No 49 |
| | R: 5′-GGAGCTGGGAGAGGATATG-3′ | | | No 50 |
| Exons-Introns part 3 | F: 5′-CTGACGCTGAAGGTTCTG-3′; | 577 bp | 50° C. | No 51 |
| | R: 5′-CAAATACATAAATAGCGACTGGGTGAC-3′ | | | No 52 |
| 3′UTR part 1 | F: 5′-CTTCCGCCAGTCATGCAAC-3′; | 582 bp | 50° C. | No 53 |
| | R: 5′-TCAAGTGATCCTCCCAACTC-3′ | | | No 54 |
| 3′UTR part 2 | F: 5′-CCTGGATGTGATTGCTCAAG-3′; | 565 bp | 50° C. | No 55 |
| | R: 5′-GGTGGAGAATGACACTCTG-3′ | | | No 56 |
| 3′UTR part 3 | F: 5′-TGAGCTGCTGGAACAAAG-3′; | 443 bp | 50° C. | No 57 |
| | R: 5′-AGCAGGCACCTTGAAATGTC-3′ | | | No 58 |

### REFERENCES

(1) Seeff L B, Hollinger F B, Alter H J, Wright E C, Cain C M, Buskell Z J, et al. Long-term mortality and morbidity of transfusion-associated non-A, non-B, and type C hepatitis: A National Heart, Lung, and Blood Institute collaborative study. Hepatology 2001 February; 33(2):455-463.
(2) Davila J A, Morgan R O, Shaib Y, McGlynn K A, El Serag H B. Hepatitis C infection and the increasing incidence of hepatocellular carcinoma: a population-based study. Gastroenterology 2004 November; 127(5):1372-1380.
(3) Thomas D L, Astemborski J, Rai R M, Anania F A, Schaeffer M, Galai N, et al. The natural history of hepatitis C virus infection: host, viral, and environmental factors. JAMA 2000 Jul. 26; 284(4):450-456.
(4) Williams R. Global challenges in liver disease. Hepatology 2006 September; 44(3):521-526.
(5) Thomas D L, Astemborski J, Rai R M, Anania F A, Schaeffer M, Galai N, et al. The natural history of hepatitis C virus infection: host, viral, and environmental factors. JAMA 2000 Jul. 26; 284(4):450-456.
(6) Lauer G M, Walker B D. Hepatitis C virus infection. N Engl J Med 2001 Jul. 5; 345(1):41-52.
(7) Manns M P, McHutchison J G, Gordon S C, Rustgi V K, Shiffman M, Reindollar R, et al. Peginterferon alfa-2b plus ribavirin compared with interferon alfa-2b plus ribavirin for initial treatment of chronic hepatitis C: a randomised trial. Lancet 2001 Sep. 22; 358(9286):958-965.
(8) Chung R T, Andersen J, Volberding P, Robbins G K, Liu T, Sherman KE, et al. Peginterferon Alfa-2a plus ribavirin versus interferon alfa-2a plus ribavirin for chronic hepatitis C in HIV-coinfected persons. N Engl J Med 2004 Jul. 29; 351(5):451-459.
(9) Fried M W, Shiffman M L, Reddy K R, Smith C, Marinos G, Goncales F L, Jr., et al. Peginterferon alfa-2a plus ribavirin for chronic hepatitis C virus infection. N Engl J Med 2002 Sep. 26; 347(13):975-982.
(10) Asselah T, Bieche I, Paradis V, Bedossa P, Vidaud M, Marcellin P. Genetics, genomics, and proteomics: implications for the diagnosis and the treatment of chronic hepatitis C. Semin Liver Dis 2007 February; 27(1):13-27.
(11) Massard J, Ratziu V, Thabut D, Moussalli J, Lebray P, Benhamou Y, et al. Natural history and predictors of disease severity in chronic hepatitis C. J Hepatol 2006; 44(1 Suppl):S19-S24.
(12) Thio C L. Host genetic factors and antiviral immune responses to hepatitis C virus. Clin Liver Dis 2008 August; 12(3):713-26, xi.
(13) Yee L J. Host genetic determinants in hepatitis C virus infection. Genes Immun 2004 June; 5(4):237-245.
(14) Kenny-Walsh E. Clinical outcomes after hepatitis C infection from contaminated anti-D immune globulin. Irish Hepatology Research Group. N Engl J Med 1999 Apr. 22; 340(16):1228-1233.
(15) Muller R. The natural history of hepatitis C: clinical experiences. J Hepatol 1996; 24(2 Suppl):52-54.
(16) Kau A, Vermehren J, Sarrazin C. Treatment predictors of a sustained virologic response in hepatitis B and C. J Hepatol 2008 October; 49(4):634-651.
(17) Grakoui A, Shoukry N H, Woollard D J, Han J H, Hanson H L, Ghrayeb J, et al. HCV persistence and immune evasion in the absence of memory T cell help. Science 2003 Oct. 24; 302(5645):659-662.
(18) Knapp S, Yee L J, Frodsham A J, Hennig B J, Hellier S, Zhang L, et al. Polymorphisms in interferon-induced genes and the outcome of hepatitis C virus infection: roles of MxA, OAS-1 and PKR. Genes Immun 2003 September; 4(6):411-419.
(19) Knapp S, Hennig B J, Frodsham A J, Zhang L, Hellier S, Wright M, et al. Interleukin-10 promoter polymorphisms and the outcome of hepatitis C virus infection. Immunogenetics 2003 September; 55(6):362-369.
(20) Wietzke-Braun P, Maouzi A B, Manhardt L B, Bickeboller H, Ramadori G, Mihm S Interferon regulatory factor-1 promoter polymorphism and the outcome of hepatitis C virus infection. Eur J Gastroenterol Hepatol 2006 September; 18(9):991-997.
(21) Yee L J, Perez K A, Tang J, van Leeuwen D J, Kaslow R A. Association of CTLA4 polymorphisms with sustained response to interferon and ribavirin therapy for chronic hepatitis C virus infection. J Infect Dis 2003 Apr. 15; 187(8):1264-1271.
(22) Goulding C, McManus R, Murphy A, MacDonald G, Barrett S, Crowe J, et al. The CCR5-delta32 mutation: impact on disease outcome in individuals with hepatitis C infection from a single source. Gut 2005 August; 54(8):1157-1161.
(23) Prasad L, Spicher V M, Zwahlen M, Rickenbach M, Helbling B, Negro F. Cohort Profile: the Swiss Hepatitis C Cohort Study (SCCS). Int J Epidemiol 2007 August; 36(4):731-737.
(24) Neumann-Haefelin C, Thimme R. Impact of the genetic restriction of virus-specific T-cell responses in hepatitis C virus infection. Genes Immun 2007 April; 8(3):181-192.
(25) Singh R, Kaul R, Kaul A, Khan K. A comparative review of HLA associations with hepatitis B and C viral infections across global populations. World J Gastroenterol 2007 Mar. 28; 13(12):1770-1787.
(26) Hoofnagle J H. Course and outcome of hepatitis C. Hepatology 2002 November; 36(5 Suppl 1):S21-S29.
(27) Thomas D L. Hepatitis C and human immunodeficiency virus infection. Hepatology 2002 November; 36(5 Suppl 1):S201-S209.
(28) Novembre J, Johnson T, Bryc K, Kutalik Z, Boyko A R, Auton A, et al. Genes mirror geography within Europe. Nature 2008 Aug. 31.
(29) Micallef J M, Kaldor J M, Dore G J. Spontaneous viral clearance following acute hepatitis C infection: a systematic review of longitudinal studies. J Viral Hepat 2006 January; 13(1):34-41.
(30) Martin-Carbonero L, Barreiro P, Jimenez-Galan G, Garcia-Berriguete R, Nunez M, Rios P, et al. Clearance of hepatitis C virus in HIV-infected patients with multiple chronic viral hepatitis. J Viral Hepat 2007 June; 14(6):392-395.
(31) Wietzke-Braun P, Manhardt L B, Rosenberger A, Uy A, Ramadori G, Mihm S Spontaneous elimination of hepatitis C virus infection: A retrospective study on demographic, clinical, and serological correlates. World J Gastroenterol 2007 Aug. 21; 13(31):4224-4229.
(32) Gale M, Jr., Foy E M. Evasion of intracellular host defence by hepatitis C virus. Nature 2005 Aug. 18; 436(7053):939-945.
(33) Sheppard P, Kindsvogel W, Xu W, Henderson K, Schlutsmeyer S, Whitmore T E, et al. IL-28, IL-29 and their class II cytokine receptor IL-28R. Nat Immunol 2003 January; 4(1):63-68.
(34) Renauld J C. Class II cytokine receptors and their ligands: key antiviral and inflammatory modulators. Nat Rev Immunol 2003 August; 3(8):667-676.
(35) Li M, Liu X, Thou Y, Su S B. Interferon-lambdas: the modulators of antivirus, antitumor, and immune responses. J Leukoc Biol 2009 July; 86(1):23-32.
(36) Kotenko S V, Gallagher G, Baurin V V, Lewis-Antes A, Shen M, Shah N K, et al. IFN-lambdas mediate antiviral protection through a distinct class II cytokine receptor complex. Nat Immunol 2003 January; 4(1):69-77.
(37) Filippini P, Coppola N, Scolastico C, Rossi G, Onofrio M, Sagnelli E, et al. Does HIV infection favor the sexual transmission of hepatitis C? Sex Transm Dis 2001 December; 28(12):725-729.
(38) Uzé G, Monneron D. IL-28 and IL-29: newcomers to the interferon family. Biochimie 2007 June-July; 89(6-7):729-34.
(39) Innis M A et al.,1990. “Academic Press”. PCR Protocols: A Guide to Methods and Applications.
(40) Bustin S A 2000). “Journal of Molecular Endocrinology, 25”. Absolute quantification of mRNA using real-time reverse transcription polymerase chain reaction assays. pp. 169-193
(41) Kwoh et al., 1990, Am. Biotechnol. Lab. 8:14-25.
(42) Hagen-Mann, et al., 1995 Exp. Clin. Endocrinol. Diabetes 103: 150-155.
(43) Walker et al., 1992 PNAS, 89:392-396.
(44) Walker et al., 1992 Nucleic Acids Res. 20: 1691-1696.
(45) Kwoh et al., 1989, PNAS 86:1173-1177.
(46) Guatelli et al., 1990, PNAS 87:1874-1878.
(47) Mueller et al., 1997, Histochem. Cell Biol. 108:431-437.
(48) Lizardi et al., 1988, BioTechnology 6:1 197-1202.
(49) Cahill et al., 1991, Clin. Chem. 37:1482-1485.
(50) Lewis, 1992, Gen. Eng. News 12 (9):1

### Equivalents

Those skilled in the art will recognize, or be able to ascertain using no more than routine experimentation, many equivalents to the specific embodiments of the invention described herein. Such equivalents are intended to be encompassed by the following claims. The contents of all references, patents and published patent applications cited throughout this application are incorporated herein by reference.

1. A method of determining a susceptibility to non-response to a hepatitis C treatment in a subject suffering from chronic hepatitis C, said method comprising determining the presence or absence of at least one polymorphic marker in the IL28B/A and/or IL-29 locus in a nucleic acid sample isolated from a biological sample obtained from said subject, wherein the presence or absence of the at least one polymorphic marker is an indication of whether the subject is susceptible to non-response to a hepatitis C treatment.

2. The method of claim 1, wherein the presence of the at least one polymorphic marker is an indication that said subject has an increased susceptibility to non-response to a hepatitis C treatment.

3. The method of claim 1, wherein the at least one polymorphic marker is located on chromosome 19 within a region comprising about 80 kb.

4. The method of claim 1, wherein the at least one polymorphic marker is located in a nucleic acid segment selected from the group of nucleic acid sequences consisting of SEQ ID No 1 to SEQ ID No 34.

5. The method of claim 1, wherein the at least one polymorphic marker is a polymorphic site associated with at least one SNP selected from the group consisting of rs11879005, rs12975799, rs11083519, rs955155, rs12972991, rs12980275, rs8105790, rs11881222, rs10853727, rs8109886, rs8113007, rs8099917, rs7248668, rs16973285, rs10853728, rs4803223, rs12980602, rs4803224, rs664893, rs576832, rs11671087, rs251910, rs7359953, rs7359950, rs2099331, rs11665818, rs570880, rs503355, rs30461, rs194014, rs251903, rs12979175, rs39587, and rs30480.

6. The method of claim 1, wherein the at least one polymorphic marker is a polymorphic site being in complete or strong linkage disequilibrium with at least one SNP selected from the group consisting of rs11879005, rs12975799, rs11083519, rs955155, rs12972991, rs12980275, rs8105790, rs11881222, rs10853727, rs8109886, rs8113007, rs8099917, rs7248668, rs16973285, rs10853728, rs4803223, rs12980602, rs4803224, rs664893, rs576832, rs11671087, rs251910, rs7359953, rs7359950, rs2099331, rs11665818, rs570880, rs503355, rs30461, rs194014, rs251903, rs12979175, rs39587, and rs30480.

7. The method of claim 1, wherein the at least one polymorphic marker is a combination of at least two SNPs selected from the group consisting of rs11879005, rs12975799, rs11083519, rs955155, rs12972991, rs12980275, rs8105790, rs11881222, rs10853727, rs8109886, rs8113007, rs8099917, rs7248668, rs16973285, rs10853728, rs4803223, rs12980602, rs4803224, rs664893, rs576832, rs11671087, rs251910, rs7359953, rs7359950, rs2099331, rs11665818, rs570880, rs503355, rs30461, rs194014, rs251903, rs12979175, rs39587, and rs30480.

8. The method of claim 1, wherein the at least one polymorphic marker is selected from the group consisting of G/T for rs8099917, G/G for rs8099917, C/G for rs576832, C/C for rs576832, G/A for rs12980275, and G/G for rs12980275.

9. The method of claim 1, wherein the hepatitis C treatment is an interferon based treatment.

10. The method of claim 9, wherein the interferon based treatment is selected from the group consisting of IFNα, IFNλ, and a pegylated-interferon.

11. The method of claim 9, wherein the interferon based treatment is combined with treatment with ribavirin, an antiprotease drug, an additional antiviral drug, and combinations thereof.

12. The method of claim 9, wherein said chronic hepatitis C is caused by a viral genotype 1, 2, 3 or 4 of HCV.

13. The method of claim 1, further comprising determining the HCV viral genotype in a nucleic acid sample isolated from a biological sample of said subject.

14. A method of determining a susceptibility to non-spontaneous hepatitis C clearance in a subject infected with hepatitis C, said method comprising determining the presence or absence of at least one polymorphic marker within the IL28B/A and/or IL-29 locus in a nucleic acid sample isolated from a biological sample obtained from said subject, wherein the presence or absence of the at least one polymorphic marker indicates a susceptibility of the subject to non-spontaneous hepatitis C clearance.

15. The method of claim 14, wherein the presence of the at least one polymorphic marker is an indication that said subject has an increased susceptibility to non-spontaneous hepatitis C clearance.

16. The method of claim 14, wherein the at least one polymorphic marker is located on chromosome 19 within a region comprising about 80 kb.

17. The method of claim 14, wherein the at least one polymorphic marker is located in a nucleic acid segment selected from the group of nucleic acid sequences consisting of SEQ ID No 1 to SEQ ID No 34.

18. The method of claim 14, wherein the at least one polymorphic marker is a polymorphic site associated with at least one SNP selected from the group consisting of rs11879005, rs12975799, rs11083519, rs955155, rs12972991, rs12980275, rs8105790, rs11881222, rs10853727, rs8109886, rs8113007, rs8099917, rs7248668, rs16973285, rs10853728, rs4803223, rs12980602, rs4803224, rs664893, rs576832, rs11671087, rs251910, rs7359953, rs7359950, rs2099331, rs11665818, rs570880, rs503355, rs30461, rs194014, rs251903, rs12979175, rs39587, and rs30480.

19. The method of claim 14, wherein said at least one polymorphic marker is a polymorphic site being in complete or strong linkage disequilibrium with at least one SNP selected from the group consisting of rs11879005, rs12975799, rs11083519, rs955155, rs12972991, rs12980275, rs8105790, rs11881222, rs10853727, rs8109886, rs8113007, rs8099917, rs7248668, rs16973285, rs10853728, rs4803223, rs12980602, rs4803224, rs664893, rs576832, rs11671087, rs251910, rs7359953, rs7359950, rs2099331, rs11665818, rs570880, rs503355, rs30461, rs194014, rs251903, rs12979175, rs39587, and rs30480.

20. The method of claim 14, wherein the polymorphic marker is a combination of at least two SNPs selected from the group consisting of rs11879005, rs12975799, rs11083519, rs955155, rs12972991, rs12980275, rs8105790, rs11881222, rs10853727, rs8109886, rs8113007, rs8099917, rs7248668, rs16973285, rs10853728, rs4803223, rs12980602, rs4803224, rs664893, rs576832, rs11671087, rs251910, rs7359953, rs7359950, rs2099331, rs11665818, rs570880, rs503355, rs30461, rs194014, rs251903, rs12979175, rs39587, and rs30480.

21. The method of claim 14, wherein the polymorphic marker is selected from the group consisting of G/T for rs8099917, G/G for rs8099917, C/G for rs576832, C/C for rs576832, G/A for rs12980275, and G/G for rs12980275.

22. The method of claim 14, wherein said chronic hepatitis C is caused by a viral genotype 1, 2, 3 or 4 of HCV.

23. The method of claim 14, further comprising determining the HCV viral genotype in a nucleic acid sample isolated from a biological sample obtained from said subject.

24. A method of treating a patient for chronic hepatitis C, comprising
determining the presence of one or more polymorphic markers in the IL28B/A and/or IL-29 locus in a nucleic acid sample isolated from a biological sample obtained from said patient, wherein the polymorphic markers is selected from the group consisting of rs11879005, rs12975799, rs11083519, rs955155, rs12972991, rs12980275, rs8105790, rs11881222, rs10853727, rs8109886, rs8113007, rs8099917, rs7248668, rs16973285, rs10853728, rs4803223, rs12980602, rs4803224, rs664893, rs576832, rs11671087, rs251910, rs7359953, rs7359950, rs2099331, rs11665818, rs570880, rs503355, rs30461, rs194014, rs251903, rs12979175, rs39587, and rs30480, and
treating the patient based upon whether the polymorphic marker is associated with increased susceptibility to non-response to hepatitis C treatment.

25. The method of claim 24, further comprising determining the HCV viral genotype of the patient, and treating the patient based upon whether the polymorphic marker and the viral genotype are associated with increased susceptibility to non-response to hepatitis C treatment.

26. The method of claim 24, wherein the hepatitis C treatment is an interferon based treatment.

27. The method of claim 26, wherein the interferon based treatment is selected from the group consisting of IFNα, IFNλ, and a pegylated-interferon.

28. The method of claim 26, wherein the interferon based treatment is combined with a treatment with ribavirin, an antiprotease drugs, an additional antiviral drugs, and combinations thereof.

29. A kit for determining a susceptibility to non-response to a hepatitis C treatment in a subject suffering from chronic hepatitis C, said kit comprising i) reagents for selectively detecting the presence or absence of at least one polymorphic marker in the IL28B/A and/or IL-29 locus in a nucleic acid sample isolated from a biological sample obtained from the subject and ii) instructions for use.

30. A kit for determining a susceptibility to non-spontaneous hepatitis C clearance in a subject infected with hepatitis C, said kit comprising i) reagents for selectively detecting the presence or absence of at least one polymorphic marker within the IL28B/A and/or IL-29 locus in a nucleic acid sample isolated from a biological sample obtained from the subject and ii) instructions for use.
